(19) United States
(12) Patent Application Publication
(10) Pub. No.: US 2024/0216545 A1
(43) Pub. Date: Jul. 4, 2024

(54) MRNA DELIVERY CONSTRUCTS AND METHODS OF USING THE SAME

(71) Applicant: GENEVANT SCIENCES GMBH, Basel (CH)

(72) Inventors: Owen DALY, Cambridge, MA (US); James HEYES, Cambridge, MA (US); Kieu LAM, Cambridge, MA (US)

(21) Appl. No.: 18/557,575

(22) PCT Filed: Apr. 28, 2022
(86) PCT No.: PCT/IB2022/053966

§ 371 Date: Oct. 26, 2023

Related U.S. Application Data
(60) Provisional application No. 63/181,115, filed on Apr. 28, 2021.

(51) Int. Cl. A61K 48/00; C12N 15/11; C12N 15/67; C12N 15/88

(52) U.S. Cl. CPC A61K 48/0066; A61K 48/0041; C12N 15/11; C12N 15/67; C12N 15/88; C12N 2800/60

(57) ABSTRACT

The present disclosure provides, among other things, polynucleotide constructs, compositions, and methods of treating a disease or disorder, including administering to a subject in need thereof a composition comprising a polynucleotide construct comprising a 5′ UTR, a mRNA encoding a protein of interest, and a 3′ UTR.

### CROSS REFERENCE TO RELATED APPLICATION

This application claims priority to U.S. Provisional Appl. No. 63/181,115 filed Apr. 28, 2021, the content of which is hereby incorporated by reference in its entirety.

### SEQUENCE LISTING

The content of the electronically submitted sequence listing in ASCII text file (Name: 4170_023 PC01_Seqlisting_ST25.txt; Size: 9,445 bytes; and Date of Creation: Apr. 21, 2022) filed with the application is incorporated herein by reference in its entirety.

### BACKGROUND

RNA molecules have the capacity to act as potent modulators of gene expression in vitro and in vivo and therefore have potential as nucleic acid based drugs. These molecules can function through a number of mechanisms utilizing either specific interactions with cellular proteins or base pairing interactions with other RNA molecules. For disorders characterized by insufficient or faulty protein production, therapeutic mRNA has the potential to provide instructions for ribosomes to produce the missing or faulty protein. Efficient and effective intracellular delivery of RNA therapeutics is difficult because these therapeutics are prone to rapid degradation and excretion in the bloodstream and do not pass freely through cell membranes.

The delivery of exogenous polynucleotides such as RNA molecules and other membrane impermeable compounds into living cells is highly restricted by the complex membrane systems of the cell. Typically, molecules used in antisense and gene therapies are large, negatively charged and hydrophilic molecules. These characteristics can preclude their direct diffusion across the cell membrane to the cytoplasm. Thus, a major barrier to the therapeutic use of polynucleotides for modulation of gene expression is the delivery of the polynucleotide to the cytoplasm. Transfection agents typically comprise peptides, polymers, and lipids of a cationic nature as well as nano- and microparticles.

These transfection agents have been used successfully in in vitro reactions. However, there are challenges with efficacy and toxicity in vivo. Furthermore, the cationic charge of these systems can cause interaction with serum components, which causes destabilization of polynucleotide-transfection reagent interaction and poor bioavailability and targeting. When transfecting nucleic acids in vivo, the delivery agent should protect the nucleic acid payload from early extracellular degradation, e.g., from nucleases. Furthermore, the delivery agent should not be recognized by the adaptive immune system (immunogenicity) and should not stimulate an acute immune response.

### BRIEF SUMMARY

The present disclosure provides polynucleotide constructs comprising, from 5′ to 3′: a 5′ UTR comprising a sequence at least 95%, 96%, 97%, 98%, 99%, or 100% identical to the sequence of SEQ ID NO: 1; an mRNA sequence comprising an open reading frame (ORF) encoding a functional protein of interest; and a 3′ UTR comprising a sequence at least 95%, 96%, 97%, 98%, 99%, or 100% identical to the sequence of SEQ ID NO: 2.

In certain aspects the disclosure provides polynucleotide constructs comprising an mRNA sequence comprising an open reading frame (ORF) encoding a functional protein of interest. In some aspects, the polynucleotide construct comprises, from 5′ to 3′: a 5′ UTR; the mRNA sequence comprising the ORF encoding the protein of interest; and a 3′ UTR. In certain aspects, the 5′ UTR comprises the sequence of SEQ ID NO: 1 and/or the 3′ UTR comprises the sequence of SEQ ID NO: 2.

In some aspects, the polynucleotide construct further comprises a 5′ terminal cap, e.g., Cap1. In some aspects, the polynucleotide construct further comprises a polyA tail. In certain aspects, the polyA tail is between 80 and 1000 nucleic acids long, e.g., between 100 and 500 nucleic acids long. A polynucleotide construct comprising, from 5′ to 3′: a 5′ terminal cap; a 5′ UTR comprising a sequence at least 99% identical to the sequence of SEQ ID NO: 1; an mRNA sequence comprising an open reading frame (ORF) encoding a functional protein of interest; a 3′ UTR comprising a sequence at least 99% identical to the sequence of SEQ ID NO: 2; and a polyA tail is between 100 and 500 nucleic acids long.

In some aspects, the mRNA comprises at least one chemically modified uridine. In certain aspects, at least about 50%, at least about 60%, at least about 70%, at least about 80%, at least about 85%, at least about 90%, at least about 95%, or about 100% of the uridines are chemically modified. In some aspects, the chemically modified uridine is selected from the group consisting of pseudouridine (ψ), N1-methyl pseudouridine (N1-me-ψ), and/or a combination thereof.

Certain aspects of the disclosure are directed to a composition comprising: a polynucleotide construct of the disclosure; and a delivery agent. In some aspects, the delivery agent comprises a lipid nanoparticle (LNP), a liposome, a polymer, a micelle, a plasmid, a virus, or any combination thereof.

In certain aspects, the LNP is selected from the group consisting of compositions within LNP1 (PEG2000-C-DMA:13-B43:Cholesterol:DSPC), LNP2 (PEG2000-S:13-B43:Cholesterol:DSPC or PEG2000-S: 18-B6:Cholesterol:DSPC), and LNP3 (PEG750-C-DLA:18-B6:Cholesterol:DSPC) groups. In some aspects, the polynucleotide construct is encapsulated in the LNP. In some aspects, the composition further comprises a pharmaceutically acceptable carrier. In some aspects, the polynucleotide construct is fully encapsulated in the LNP. In some aspects, at least 91%, at least 92%, at least 93%, at least 94%, at least 95%, at least 96%, at least 97%, at least 98%, at least 99%, or more of the polynucleotide construct is encapsulated by the LNP.

Certain aspects of the disclosure are directed to a method for increasing the expression of a protein of interest in a cell comprising administering to the cell a composition comprising a polynucleotide construct of the disclosure or the composition of the disclosure.

Certain aspects of the disclosure are directed to a method for treating or reducing the symptoms associated with a disease or disorder comprising administering to a subject in need thereof a therapeutically effective amount of a composition comprising the polynucleotide construct of the disclosure or the composition of the disclosure.

Certain aspects of the disclosure are directed to an expression cassette comprising a polynucleotide construct comprising, from 5′ to 3′: a 5′ UTR comprising the sequence of SEQ ID NO: 1; an mRNA sequence comprising an open reading frame (ORF) encoding a functional protein of interest; and a 3′ UTR comprising the sequence of SEQ ID NO: 2. In some aspects, the expression cassette further comprises a promoter, e.g., a T7 promoter.

Some aspects of the disclosure are directed to a plasmid comprising the expression cassette of the disclosure. In some aspects, the expression cassette transcribes an mRNA of the disclosure. Some aspects of the disclosure are directed to a host cell comprising an expression cassette of the disclosure, or the plasmid of the disclosure.

Certain aspects of the disclosure are directed to use of the polynucleotide construct of the disclosure, or the composition of the disclosure, or the expression cassette of the disclosure, or the plasmid of the disclosure, or the host cell of the disclosure, for the manufacture of a medicament for the treatment of a disease or disorder in a subject in need thereof.

Certain aspects of the disclosure are directed to methods for the in vivo delivery of a nucleic acid, the method comprising: administering to a mammalian subject a polynucleotide construct of the disclosure, or a composition of the disclosure, or an expression cassette of the disclosure, or a plasmid of the disclosure, or a host cell of the disclosure.

Certain aspects of the disclosure are directed to methods for treating a disease or disorder in a mammalian subject in need thereof, the method comprising: administering to the mammalian subject a therapeutically effective amount of a polynucleotide construct of the disclosure, or a composition of the disclosure, or an expression cassette of the disclosure, or a plasmid of the disclosure, or a host cell of the disclosure.

In some aspects, the disease or disorder is a genetic disease or disorder. In some aspects, the disease or disorder is an infectious disease or a cancer.

In some aspects, the protein of interest comprises an enzyme, a growth factor, a cytokine, a receptor, a receptor ligand, a hormone, a membrane protein, a membrane-associated protein, an antigen or an antibody. In some aspects, the protein of interest is an enzyme.

These and other aspects will be apparent from a reading of the following detailed description.

### BRIEF DESCRIPTION OF THE FIGURES

In some instances, the disclosure can be more completely understood in consideration of the following detailed description of various aspects of the disclosure in connection with the accompanying Figures, in which:

FIG. 1 shows MCP-1 induction at 6 hours after the first dose in rats administered LNP encapsulating mRNA constructs (Cap1-5′ UTR (SEQ ID NO: 1)-OTC mRNA-3′UTR (SEQ ID NO: 2)-polyA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 2A shows MCP-1 induction at 6 hours after the first, second, and third dose on Day 0, 7, and 14 respectively, in rats administered LNP encapsulating mRNA constructs (Cap1-5′ UTR (SEQ ID NO: 1)-OTC mRNA-3′UTR (SEQ ID NO: 2)-polyA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 2B shows IP-1 induction at 6 hours after the first, second, and third dose on Day 0, 7 and 14 respectively, in rats administered LNP encapsulating mRNA constructs (Cap1-5′ UTR (SEQ ID NO: 1)-OTC mRNA-3′UTR (SEQ ID NO: 2)-polyA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 3A shows hOTC protein expression in rat livers after a single dose administration of LNP encapsulating mRNA constructs (Cap1-5′ UTR (SEQ ID NO: 1)-OTC mRNA-3′UTR (SEQ ID NO: 2)-polyA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 3B shows hOTC protein expression in rat livers after a single versus multi-dose administration of LNP carrying mRNA constructs (Cap1-5′ UTR (SEQ ID NO: 1)-OTC mRNA-3′UTR (SEQ ID NO: 2)-polyA) having different poly(A) tail lengths (80, 161, 208, 262, 322, or 440 nucleotides) compared to PBS control. The 80 nucleotide poly(A) was encoded and the other tested poly(A) were enzymatic (enz).

FIG. 4 shows MCP-1 induction at 6 hours after the first dose in mice administered with LNP1 or LNP2 (ionizable lipid: 13-B43) groups encapsulating mRNA constructs (Cap1-5′ UTR (SEQ ID NO: 1)-OTC mRNA-3′UTR (SEQ ID NO: 2)-polyA) with different modifications: PsU, N1MePsU, or 5MoU, compared to PBS control.

FIG. 5 shows hOTC expression at 24 hours post dose in mice administered with LNP1 or LNP2 (ionizable lipid: 13-B43) groups encapsulating mRNA constructs (Cap1-5′ UTR (SEQ ID NO: 1)-OTC mRNA-3′UTR (SEQ ID NO: 2)-polyA) with different modifications: PsU, N1MePsU, or SMOU, compared to PBS control.

FIG. 6A shows anti-PEG IgG antibody response in rats administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) compared to EPO and Luc payloads.

FIG. 6B shows anti-PEG IgM antibody response in rats administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) compared to EPO and Luc payloads.

FIG. 7 shows MCP-1 induction at 6 hours in rats administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) compared to EPO and Luc payloads and PBS at 0, 7 and 14 days.

FIG. 8 shows OTC protein expression in rats administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) after 1 and 3 doses.

FIG. 9 shows lipid concentration (clearance) in rat livers following administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) after 1 and 3 doses.

FIG. 10A shows ALT levels in rats following administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) after 1 and 3 doses.

FIG. 10B shows AST levels in rats following administered different LNP (LNP1, LNP2 (ionizable lipid: 13-B43), LNP2 (ionizable lipid: 18-B6), or LNP3) groups encapsulating mRNA constructs (Cap1-5′ UTR (SEQ ID NO: 1)-OTC mRNA-3′UTR (SEQ ID NO: 2)-polyA) after 1 and 3 doses.

FIG. 11A-11C shows cytokine response following administration of an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA) after weekly repeat doses. FIG. 11A shows MCP-1 induction 6 hours post dose, FIG. 11B shows IP-10 induction 6 hours post dose, and FIG. 11C shows MIP-la induction 6 hours post dose.

FIG. 12 shows anti-PEG IgM antibody response following administration of LNP2 (ionizable lipid: 13-B43) encapsulating mRNA constructs (Cap1-5′ UTR (SEQ ID NO: 1)-OTC mRNA-3′UTR (SEQ ID NO: 2)-polyA) after weekly repeat doses compared to PBS control.

FIG. 13 shows anti-PEG IgG antibody response following administration of an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA) after weekly repeat doses compared to PBS control.

FIG. 14 shows anti-OTC IgM antibody response following administration of an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA) after weekly repeat doses compared to PBS control.

FIG. 15 shows anti-OTC IgM antibody response following administration of an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA) after weekly repeat doses compared to PBS control.

FIG. 16 shows OTC protein expression in rats administered an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA) after weekly repeat doses.

FIG. 17A-17B show human OTC mRNA (hOTC mRNA) in (A) liver and (B) plasma of rats administered an LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA).

FIG. 18A shows the average ALT levels 24 hours post-dose in the liver of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 18B shows the average AST levels 24 hours post-dose in the liver of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 18C shows the individual (R1, R2, or R3) and average ALT levels 24 hours post-dose in the liver of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 18D shows the individual (R1, R2, or R3) and average AST1 levels 24 h post-dose in the liver of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIGS. 19A-19D shows (A) the average GGT levels, (B) total bilirubin levels, (C) individual (R1, R2, or R3) and average GGT levels, and (D) individual (R1, R2, or R3) and average total bilirubin levels 24 hours post-dose of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 20A-20C shows (A) the neutrophil levels, (B) the monocyte levels, and (C) the platelet levels at 24 hours post-dose of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 21A-21C shows (A) the MCP-1 levels, (B) the MIP-la levels, and (C) the IP-10 levels at 6 hours post-dose of rats administered an LNP1, LNP2 (ionizable lipid: 13-B43) or LNP2 (ionizable lipid: 18-B6) composition encapsulating mRNA constructs (Cap1-5′ UTR (SEQ ID NO: 1)-OTC mRNA-3′UTR (SEQ ID NO: 2)-polyA).

FIG. 22 shows OTC expression at 24 hours post-dose of rats administered an LNP1 or LNP2 (ionizable lipid: 13-B43) composition encapsulating mRNA constructs (Cap1-5′ UTR (SEQ ID NO: 1)-OTC mRNA-3′UTR (SEQ ID NO: 2)-polyA).

FIGS. 23A-23C show (A) human OTC (hOTC), (B) MCP-1, and (C) IL-6 protein expression levels in the livers of non-human primates that were administered LNP1 encapsulating mRNA constructs (Cap1-5′ UTR (SEQ ID NO: 1)-OTC mRNA-3′UTR (SEQ ID NO: 2)-polyA) at 0.25 mg/kg. 1 mg/kg, and 3 mg/kg. The hOTC protein expression is shown as % of endogenous, and the MCP-1 and IL-6 protein expression are shown compared to 0 mg/kg control.

FIGS. 24A-24B shows (A) hEPO expression and (B) MCP-1 induction in mice that were administered LNP1 encapsulating mRNA constructs (Cap-5′ UTR (SEQ ID NO: 1)-hEPO mRNA (SEQ ID NO: 4)-3′ UTR (SEQ ID NO: 2)-polyA).

FIGS. 25A-25B shows (A) hMMP-8 and (B) IL-6 induction in mice that were administered LNP1 encapsulating mRNA constructs (Cap-5′ UTR (SEQ ID NO: 1)-hMMP-8 mRNA (SEQ ID NO: 5)-3′ UTR (SEQ ID NO: 2)-polyA).

FIG. 26 shows anti-ovalbumin titers in mice that were administered LNP1 encapsulating mRNA constructs (Cap-5′ UTR (SEQ ID NO: 1)-2-M9 mRNA (SEQ ID NO: 6)-3′ UTR (SEQ ID NO: 2)-polyA) and (Cap-5′ UTR (SEQ ID NO: 1)-2-M10 mRNA (SEQ ID NO: 7)-3′ UTR (SEQ ID NO: 2)-polyA).

FIGS. 27A-27B shows (A) anti-hemagglutinin titers and (B) hemagglutinin inhibition in mice that were administered LNP1 encapsulating mRNA constructs (Cap-5′ UTR (SEQ ID NO: 1)-2-M6-HA (SEQ ID NO: 8)-3′ UTR (SEQ ID NO: X)-polyA).

### DETAILED DESCRIPTION

The present disclosure is directed to improved constructs comprising polynucleotides (e.g., mRNA), compositions, and methods for expressing polynucleotides (e.g., mRNA) in a cell and use of such constructs, polynucleotides and compositions. Unless defined otherwise, all technical and scientific terms used herein have the same meaning as commonly understood by one of ordinary skill in the art pertinent to the methods and compositions described. The definitions provided herein are to facilitate understanding of certain terms used frequently herein.

As used in this specification and the appended claims, the singular forms “a”, “an”, and “the” encompass aspects having plural referents, unless the content clearly dictates otherwise.

As used herein, the term “nucleic acid,” in its broadest sense, refers to any compound and/or substance that is or can be incorporated into a polynucleotide chain, e.g., via a phosphodiester linkage. In some aspects, “nucleic acid” refers to individual nucleic acid residues (e.g., nucleotides and/or nucleosides). In some aspects, “nucleic acid” refers to a polynucleotide chain comprising individual nucleic acid residues. In some aspects, “nucleic acid” encompasses RNA, e.g., mRNA, as well as single and/or double-stranded DNA and/or cDNA.

As used herein the term “polynucleotide” or “oligonucleotide” refers to a polymer comprising 7-20,000 nucleotide monomeric units (i.e., from 7 nucleotide monomeric units to 20,000 nucleotide monomeric units, inclusive). Polynucleotides include deoxyribonucleic acid (DNA) and ribonucleic acid (RNA), or their derivatives, and combinations of DNA and RNA. For example, DNA can be in form of cDNA, in vitro polymerized DNA, plasmid DNA, parts of a plasmid DNA, expression vectors, expression cassettes, chimeric sequences, recombinant DNA, chromosomal DNA, or any derivatives thereof. In further examples, RNA can be in the form of messenger RNA (mRNA), in vitro polymerized RNA, recombinant RNA, transfer RNA (tRNA), small nuclear RNA (snRNA), ribosomal RNA (rRNA), chimeric sequences, recombinant RNA, or any derivatives thereof. In addition, DNA and RNA can be single, double, triple, or quadruple stranded.

Further examples of polynucleotides as used herein include, but are not limited to single stranded mRNA, which can be modified or unmodified. Modified mRNA includes those with at least two modifications and a translatable region. The modifications can be located on the backbone and/or a nucleoside of the nucleic acid molecule. The modifications can be located on both a nucleoside and a backbone linkage.

As used herein, the term “messenger RNA” or “mRNA” refers to a polyribonucleotide that encodes at least one polypeptide. mRNA as used herein encompasses both modified and unmodified RNA. mRNA can contain one or more coding and non-coding regions. mRNA can be purified from natural sources, produced using recombinant expression systems and optionally purified, in vitro transcribed, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some aspects, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, “expression” of a nucleic acid sequence refers to translation of a polynucleotide, e.g., an mRNA, into a polypeptide, assembly of multiple polypeptides into an intact protein (e.g., enzyme) and/or post-translational modification of a polypeptide or fully assembled protein (e.g., enzyme). In this disclosure, the terms “expression” and “production,” and grammatical equivalent, are used inter-changeably.

As used herein, the term “amino acid,” in its broadest sense, refers to any compound and/or substance that can be incorporated into a polypeptide chain. In some aspects, an amino acid has the general structure H₂N—C(H)(R)—COOH. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids can participate in a disulfide bond. Amino acids can comprise one or posttranslational modifications, such as association with one or more chemical entities (e.g., methyl groups, acetate groups, acetyl groups, phosphate groups, formyl moicties, isoprenoid groups, sulfate groups, polyethylene glycol moieties, lipid moieties, carbohydrate moieties, biotin moieties, etc.). The term “amino acid” is used interchangeably with “amino acid residue.” and can refer to a free amino acid and/or to an amino acid residue of a peptide. It will be apparent from the context in which the term is used whether it refers to a free amino acid or a residue of a peptide.

A “polypeptide” is a polymer of amino acid residues joined by peptide bonds, whether produced naturally or synthetically.

As used herein the term “peptide” refers to a polypeptide having 2-100 amino acid monomers.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Some proteins are defined herein in terms of their amino acid backbone structures.

A “protein of interest” is a protein or peptide whose expression is desired. In some aspects, the protein of interest is a wild-type protein. In some aspects, the protein of interest is modified relative to wild-type protein.

As used herein, a “functional” biological molecule, e.g., a protein of interest, is a biological molecule in a form in which it exhibits a property and/or activity by which it is characterized.

As used herein, the term “delivery” encompasses both local and systemic delivery. For example, delivery of a polynucleotide, e.g., an mRNA, encompasses situations in which a polynucleotide is delivered to a target tissue and the encoded protein is expressed and retained within the target tissue (also referred to as “local distribution” or “local delivery”). Other exemplary situations include one in which a polynucleotide is delivered to a target tissue and the encoded protein is expressed and secreted into patient's circulation system (e.g., serum) and systematically distributed and taken up by other tissues (also referred to as “systemic distribution” or “systemic delivery). In other exemplary situations, a polynucleotide is delivered systemically and is taken up in a wide variety of cells and tissues in vivo. In some exemplary situations, the delivery is intravenous, intramuscular or subcutaneous.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term can be used to refer to events that occur within a living cell (as opposed to, for example, in vitro systems).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein the term “treating” refers to the administration of a delivery agent and nucleic acid that eliminates, alleviates, inhibits the progression of, or reverses progression of, in part or in whole, any one or more of the pathological hallmarks or symptoms of any one of the diseases and disorders being treated. In some aspect, the disease can be a disease caused by a deficiency in a protein of interest. In some aspects, the disease can be an infectious disease or cancer. The phrase “therapeutically effective” as used herein is intended to qualify the amount of polynucleotide or pharmaceutical composition, or the combined amount of active ingredients in the case of combination therapy. This amount or combined amount will achieve the goal of treating the relevant disease or condition.

As used herein, the term “subject” refers to a human or any non-human animal (e.g., mouse, rat, rabbit, dog, cat, cattle, swine, sheep, horse or primate). A human includes pre- and post-natal forms. In many aspects, a subject is a human. A subject can be a patient, which refers to a human presenting to a medical provider for diagnosis or treatment of a disease. The term “subject” can be used herein interchangeably with “individual” or “patient.” A subject can be afflicted with or is susceptible to a disease or disorder but may or may not display symptoms of the disease or disorder.

The term “lipid” refers to a group of organic compounds that are esters of fatty acids and are characterized by being insoluble in water but soluble in many organic solvents. They are usually divided in at least three classes: (1) “simple lipids” which include fats and oils as well as waxes; (2) “compound lipids” which include phospholipids and glycolipids; (3) “derived lipids” such as steroids.

The term “amphipathic lipid” refers, in part, to any suitable material wherein the hydrophobic portion of the lipid material orients into a hydrophobic phase, while a hydrophilic portion orients toward the aqueous phase. Amphipathic lipids are usually the major component of a lipid LNP. Hydrophilic characteristics derive from the presence of polar or charged groups such as carbohydrates, phosphato, carboxylic, sulfato, amino, sulfhydryl, nitro, hydroxy and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid, glycosphingolipid families, diacylglycerols and ß-acyloxyacids, are also within the group designated as amphipathic lipids. Additionally, the amphipathic lipid described above can be mixed with other lipids including triglycerides and sterols.

The term “anionic lipid” refers to any lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

The term “cationic lipid” refers to any of a number of lipid species which carry a net positive charge at a selective pH, such as physiological pH. Such lipids include, but are not limited to, N, N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-diolcoyloxy)propyl)-N,N,N-trimethylammonium chloride (“DOTAP”); 3-(N—(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present disclosure. These include, for example, LIPOFECTIN® (commercially available cationic liposomes comprising DOTMA and 1,2-diolcoyl-sn-3-phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (“DOSPA”) and (“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA and the like.

The term “lipid nanoparticle” refers to any lipid composition that can be used to deliver a compound (e.g., a polynucleotide construct) including, but not limited to, liposomes, wherein an aqueous volume is encapsulated by an amphipathic lipid bilayer; or wherein the lipids coat an interior comprising a large molecular component, such as a plasmid, with a reduced aqueous interior; or lipid aggregates or micelles, wherein the encapsulated component is contained within a relatively disordered lipid mixture.

As used herein, “lipid encapsulated” or “lipid encapsulation” can refer to a lipid formulation which provides a compound (e.g., a polynucleotide construct) with full encapsulation, partial encapsulation, or both. “Full encapsulation” or “fully encapsulated” is understood herein to mean at least 90% a compound (e.g., a polynucleotide construct) in a lipid formulation is encapsulated by the lipid (e.g., LNP). In some aspects, at least 95%, at least 96%, at least 97%, at least 98%, at least 99%, or more of the compound (e.g., a polynucleotide construct) in a lipid formulation is encapsulated by the lipid (e.g., LNP).

As used herein, the term “5′-terminal untranslated region,” “5′-UTR,” or “5′UTR” refers to a nucleic sequence that is not translated into a protein and is located at the 5′ end of the coding sequence.

As used herein, the term “3′-terminal untranslated region,” “3′-UTR,” or “3′UTR” refers to a nucleic acid sequence that is located at the 3′ end of the coding sequence, typically between the mRNA sequence encoding a protein of interest (open reading frame (ORF) or coding sequence (CDS)) and a poly(A) sequence.

As used herein, the term “5′ terminal cap” or “5′ cap” refers to a chemical modification that is incorporated at the 5′ terminus of an mRNA. The 5′ terminal cap can protect the nucleic acid molecule from exonuclease degradation, and can help in delivery and/or localization within a cell.

### Polynucleotide Constructs

The polynucleotide constructs disclosed herein can be used as therapeutic agents to increase the level of a protein of interest in a cell (in vitro or in vivo) to a level greater than that obtained and/or observed in the absence of the polynucleotide constructs disclosed herein.

In certain aspects, the polynucleotide construct comprises a nucleic acid sequence, e.g., an mRNA sequence, comprising an open reading frame (ORF) encoding a functional protein or peptide. The ORF can encode a full length protein or a functional fragment thereof.

In some aspects, the polynucleotide construct comprises an mRNA sequence comprising an ORF which is codon optimized. The mRNA can encode any protein or peptide of interest that is capable of being expressed in a cell. Exemplary proteins or peptides encoded by the mRNA include, but are not limited to, enzymes, growth factors, cytokines, receptors, receptor ligands, therapeutic proteins, hormones, membrane proteins, membrane-associated proteins, antigens, and antibodies.

In some aspect, length of the mRNA coding for the protein of interest is greater than about 30 nucleotides in length. In some aspects, the mRNA coding for the protein of interest is greater than 30, 35, 40, 45, 50, 60, 75, 100, 125, 150, 175, 200, 250, 300, 350, 400, 450, 500, 600, 700, 800, 900, 1000, 1100, 1200, 1300, 1400, 1500, 1800, 2000, 3000, 4000, 5000 nucleotides, or greater than 5000 nucleotides. In some aspect, length of the mRNA is 30 to 5000, 30 to 4000, 30 to 3000, or 30 to 2000 nucleotides in length. In some aspects, the mRNA is 30 to 5000, 35 to 5000, 40 to 5000, 45 to 5000, 50 to 5000, 60 to 5000, 75 to 5000, 100 to 5000, 125 to 5000, 150 to 5000, 175 to 5000, 200 to 5000, 250 to 5000, 300 to 5000, 350 to 5000, 400 to 5000, 450 to 5000, 500 to 5000, 600 to 5000, 700 to 5000, 800 to 5000, 900 to 5000, 1000 to 5000, 1100 to 5000, 1200 to 5000, 1300 to 5000, 1400 to 5000, 1500 to 5000, 1800 to 5000, 2000 to 5000, 3000 to 5000, 4000 to 5000, 5000 to 6000 nucleotides, or greater than 5000 nucleotides.

In some aspects, the protein of interest encoded by the mRNA is an enzyme. In some aspects, the protein of interest is an enzyme selected from ornithine transcarbamylase (OTC), Erythropoietin (EPO), argininosuccinate lyase (ASL), or matrix metalloproteinase-8 (MMP-8). In some aspects, the protein of interest is an enzyme selected from Erythropoietin (EPO) or argininosuccinate lyase (ASL). In some aspects, the protein of interest is Erythropoietin (EPO), e.g., human EPO (hEPO). In some aspects, the protein of interest is argininosuccinate lyase (ASL). In some aspects, the protein of interest is matrix metalloproteinase-8 (MMP-8), e.g., human MMP-8 (hMMP-8). In some aspects, the protein of interest is not ornithine transcarbamylase (OTC).

In some aspects, the protein of interest is an antigen selected from a SARS-COV2 protein (e.g., SARS-COV2 spike protein) and an influenza protein (e.g., Hemagglutinin (HA)).

The protein or peptide of interest can be any protein capable of being expressed in a cell. In some aspects, the constructs, polynucleotides, or compositions of the disclosure are delivered to a cell resulting in the expression of a protein of interest, e.g., enzymes, growth factors, cytokines, receptors, receptor ligands, therapeutic proteins, hormones, membrane proteins, membrane-associated proteins, antigens, or antibodies.

In some aspects, the polynucleotide construct comprises a 5′ UTR. In some aspects, the 5′ UTR is between about 10 and about 100, about 20 and about 80, about 30 and about 60, or about 40 and about 50 nucleotides in length. In some aspects, the 5′ UTR is between about 40 and about 50 nucleotides in length.

In some aspects, the 5′ UTR has a nucleic acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 1. In some aspects, the 5′ UTR has the nucleic acid sequence of SEQ ID NO: 1.

In some aspects, the polynucleotide construct comprises a 3′ UTR.

In some aspects, the 3′ UTR is between about 10 and about 200, about 40 and about 180, about 60 and about 160, about 80 and about 140, about 100 and about 120 nucleotides in length. In some aspects, the 3′ UTR is between about 100 and about 120 nucleotides in length.

In some aspects, the 3′ UTR has a nucleic acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2. In some aspects, the 3′ UTR has the nucleic acid sequence of SEQ ID NO: 2.

In some aspects, a polynucleotide construct of the disclosure comprises, from 5′ to 3′: (i) a 5′ UTR, e.g., comprising the sequence of SEQ ID NO: 1; (ii) a nucleic acid sequence, e.g., a mRNA, comprising an open reading frame (ORF) encoding a protein of interest; and a 3′ UTR comprising the sequence of SEQ ID NO: 2.

The polynucleotide construct can further comprise a polyA tail. In some aspects, the polyA tail is a 3′-poly (A) tail comprising a monotonous portion of the adenine nucleotide sequence at the 3′-end of the transcribed mRNA. In some aspects, the polyA tail can include up to about 500 adenine nucleotides. In some aspects, the length of the polyA tail enhances the stability of the mRNA. In some aspects, the polyA tail is longer than 80, 81, 82, 83, 84, 85, 86, 87, 88, 89, 90, 91, 92, 93, 94, 95, 96, 97, 98, 99, 100, 110, 115, 120, 125, 130, 135, 140, 145, or 150 nucleic acids. In some aspects, the polyA tail is between 80 to 1000, 85 to 1000, 90 to 1000, 95 to 1000, 100 to 1000, 105 to 1000, 110 to 1000, 115 to 1000, 120 to 1000, 125 to 1000, 130 to 1000, 135 to 1000, 140 to 1000, 145 to 1000, 150 to 1000, 155 to 1000, 160 to 1000, 80 to 800, 85 to 800, 90 to 800, 95 to 800, 100 to 800, 105 to 800, 110 to 800, 115 to 800, 120 to 800, 125 to 800, 130 to 800, 135 to 800, 140 to 800, 145 to 800, 150 to 800, 155 to 800, or 160 to 800 nucleic acids long. In some aspects, the polyA tail is between 100 and 500 nucleic acids long.

In some aspects, the polynucleotide construct further comprises a 5′ terminal cap. In some aspects, the 5′ terminal cap is selected from the group consisting of Cap0, Cap1. ARCA, inosine, N1-methyl-guanosine, 2′ fluoro-guanosine, 7-deaza-guanosine, 8-oxo-guanosine, 2-amino-guanosine, LNA-guanosine, 2-azido-guanosine, Cap2 and Cap4. In some aspects, the 5′ terminal cap is Cap1.

In some aspects, the polynucleotide construct comprises a start codon at the 5′ end of the ORF. In some aspects, the polynucleotide construct comprises a stop codon at the 3′ end of the ORF.

In some aspects, the polynucleotide construct comprises a 5′ terminal cap, a 5′ UTR, an open reading frame (ORF) encoding a protein of interest, a 3′ UTR, and a poly(A). In some aspects, the polynucleotide construct comprises Cap1, a 5′ UTR having the nucleic acid sequence of SEQ ID NO: 1, an open reading frame (ORF) encoding a protein of interest, a 3′ UTR having the nucleic acid sequence of SEQ ID NO: 1, and a poly(A).

In some aspects, the polynucleotide construct comprises in the 5′ to 3′ direction: a 5′ terminal cap, a 5′ UTR, an open reading frame (ORF) encoding a protein of interest, a 3′ UTR, and a poly(A). In some aspects, the polynucleotide construct comprises in the 5′ to 3′ direction: Cap1, a 5′ UTR having the nucleic acid sequence of SEQ ID NO: 1, an open reading frame (ORF) encoding a protein of interest, a 3′ UTR having the nucleic acid sequence of SEQ ID NO: 1, and a poly(A).

In certain aspects, the polynucleotide construct comprises a modified nucleotide. In some aspects, the polynucleotide construct comprises an mRNA sequence comprising an open reading frame (ORF) encoding a functional protein of interest, wherein the mRNA sequence comprises a modified nucleotide. In some aspects, the modified nucleotide is uridine. In some aspects, at least about 50%, at least about 60%, at least about 70%, at least about 80%, at least about 90%, at least about 95%, at least about 99%, or 100% of the uridines are chemically modified.

In some aspects, the chemically modified uridine is selected from the group consisting of pseudouridine (w), N1-methyl pseudouridine (N1-me-ψ), 5-methoxy uridine (5moU), and any combination thereof. In some aspects, the chemically modified uridine is selected from the group consisting of pseudouridine (w), N1-methyl pseudouridine (N1-me-ψ), and any combination thereof. In certain aspects, the ORF comprises at least 95%, at least 98%, at least 99%, or about 100% modified uridines, e.g., pseudouridine (v) modified or N1-methyl pseudouridine (N1-me-ψ) modified.

In some aspects, the expression cassette further comprises a promoter. In some aspects, the promoter is a T7 promoter. In some aspects, the T7 promoter comprises the following 5′ to 3′ sequence: TAATACGACTCACTATA (SEQ ID NO: 3). In some aspects, the 5′ UTR of the expression cassette comprises an adenine (A) immediately downstream of the promoter, e.g., T7 promoter. Some aspects are directed to a plasmid comprising the expression cassette. In some aspects, the plasmid further comprises an antibiotic resistance gene. In some aspects, the polynucleotide construct is prepared using in vitro transcription.

Exemplary nucleic acid sequences of polynucleotide components are shown in Table 1 herein.

TABLE 1
Sequence Related to Polynucleotide Constructs

| SEQ | | |
|---|---|---|
| ID NO: | Sequence | Description |
| 1 | AGGAAAUAAGAGAGAAAAGAAGAGUAAGAA | 5′ UTR |
| | GAAAUAUAAGAGCCACC | |
| 2 | GCGGCCGCUUAAUUAAGCUGCCUUCUGCGG | 3′ UTR |
| | GGCUUGCCUUCUGGCCAUGCCCUUCUUCUC | |
| | UCCCUUGCACCUGUACCUCUUGGUCUUUGA | |
| | AUAAAGCCUGAGUAGGAAGUCUAG | |
| 3 | TAATACGACTCACTATA | T7 Promoter |

In some aspects, the polynucleotide construct of the disclosure is formulated with a delivery agent, e.g., a lipid nanoparticle (LNP).

### Delivery Agents

The delivery agents disclosed herein can effectively transport the polynucleotide constructs, cassettes, and mRNA disclosed herein into cells in vitro and in vivo.

In certain aspects, the delivery agent is a lipid nanoparticle, a liposome, a polymer, a micelle, a plasmids, a viral deliver agent, or any combination thereof.

Without being bound to any particular theory, the transport of polynucleotides constructs, expression cassettes, and/or mRNA disclosed herein by a delivery agents can occur via delivery of the polynucleotide construct to the cytosol of a cell. As gene expression and mRNA translation occurs in the cytosol of a cell, the polynucleotides have to enter the cytosol for effective modulation of the target gene or effective translation of a transported mRNA. If the polynucleotides do not enter the cytosol, they are likely to either be degraded or remain in the extracellular medium.

Examples of methods for the intracellular delivery of a biologically active polynucleotide to a target cell include those where the cell is in a mammalian animal, including, for example, a human, rodent, murine, bovine, canine, feline, sheep, equine, and simian mammal. In some aspects, the target cells for intracellular delivery are liver cells.

In some aspects, the delivery agent is a lipid nanoparticle (LNP). The polynucleotide constructs of the disclosure can be formulated within a LNP. In certain aspects, the polynucleotide construct is encapsulated within the LNP. “Encapsulated” as used herein refers containing a molecule, e.g., a polynucleotide, within the interior space of the LNP. In some aspects, by encapsulating the polynucleotide construct (e.g., comprising mRNA) within a delivery agent, such as a LNP, the nucleic acid (e.g., the polynucleotide construct of the disclosure) can be protected from an environment, which can contain enzymes or chemicals that degrade nucleic acids and/or systems or receptors that cause the rapid excretion of the nucleic acids. Lipid nanoparticles typically comprise an ionizable (e.g., cationic) lipid, a non-cationic lipid (e.g., cholesterol and a phospholipid), and a PEG lipid (e.g., a conjugated PEG lipid), which can be formulated with a payload of interest, e.g., a polynucleotide construct disclosed herein. The polynucleotide construct, e.g., mRNA, of the disclosure can be encapsulated in the lipid particle, thereby protecting it from enzymatic degradation. In some aspects, the molecule (e.g., a polynucleotide construct) is fully encapsulated by the LNP. In some aspects, at least 95%, at least 96%, at least 97%, at least 98%, at least 99%, or more of the molecule (e.g., a polynucleotide construct) in a lipid formulation is encapsulated by the LNP.

Certain aspects are directed to a composition comprising: a polynucleotide construct of the disclosure; and a delivery agent. The delivery agent can comprise an LNP, e.g., LNP compositions in LNP1 (PEG2000-C-DMA:13-B43:Cholesterol:DSPC), LNP2 (PEG2000-S:13-B43:Cholesterol:DSPC or PEG2000-S:18-B6:Cholesterol:DSPC), or LNP3 (PEG750-C-DLA:18-B6:Cholesterol:DSPC) groups.

In some aspects, the LNP of the disclosure comprises a PEG lipid selected from the group consisting of PEG2000-C-DMA, PEG2000-S, and PEG750-C-DLA. In some aspects, the LNP comprises a PEG lipid which is PEG2000-C-DMA. In some aspects, the LNP comprises a PEG lipid which is PEG2000-S. In some aspects, the LNP comprises a PEG lipid which is PEG750-C-DLA.

In some aspects, the LNP of the disclosure comprises an ionizable lipid which is 13-B43 or 18-B6.

In some aspects, the ionizable lipid is a compound of formula 13-B43, or a salt thereof. Such lipids are described, e.g., in WO 2013/126803 (PCT/US2013/027469).

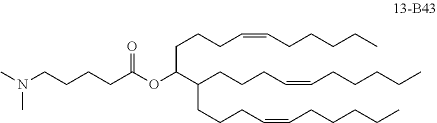 [SMILES: CCCCC/C=C\CCCC(CCC/C=C\CCCCC)C(CCC/C=C\CCCCC)OC(=O)CCCCN(C)C]

In some aspects, the ionizable lipid is a compound of formula 18-B6, or a salt thereof.

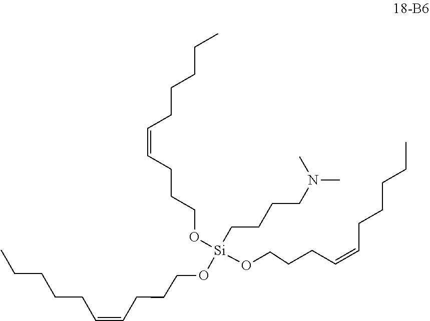 [SMILES: CCCCC/C=C\CCCO[Si](CCCCN(C)C)(OCCC/C=C\CCCCC)OCCC/C=C\CCCCC]

In some aspects, the LNP of the disclosure comprises a non-cationic lipid. In certain aspects the non-cationic lipid is a cholesterol, Distearoyl phosphatidylcholine (DSPC), or a combination thereof. In some aspects, the LNP comprises cholesterol. In some aspects, the LNP comprises Distearoyl phosphatidylcholine (DSPC). In some aspects, the LNP comprises cholesterol and Distearoyl phosphatidylcholine (DSPC).

In some aspects, the LNP of the disclosure comprises (a) a PEG Lipid (e.g. PEG2000-C-DMA, PEG2000-S, or PEG750-C-DLA); (b) an ionizable lipid (13-B43 or 18-B6); (c) a cholesterol; and (d) Distearoyl phosphatidylcholine (DSPC).

In certain aspects, the LNP of the disclosure comprises a PEG lipid in an amount of 0.1-4 mol %; 0.5-4 mol %; 2-3.5 mol %, 0.1-2 mol %; 0.5-2 mol %, or 1-2 mol % of the LNP. In certain aspects, the LNP comprises an ionizable lipid in an amount of 50-85 mol %; 50-65 mol %, or 50-60 mol % of the LNP. In certain aspects, the LNP comprises a non-cationic lipid in an amount of 45-50 mol % or up to about 50 mol %. In certain aspects, the LNP comprises a cholesterol in an amount of 30-40 mol % or 30-35 mol % of the LNP. In certain aspects, the LNP comprises an DSPC in an amount of 3-15 mol % or 6-12 mol % of the LNP.

In some aspects, the LNP of the disclosure comprises (a) 1-4 mol % PEG Lipid (e.g. PEG2000-C-DMA, PEG2000-S, or PEG750-C-DLA); (b) 50-60 mol % ionizable lipid (13-B43 or 18-B6); and (c) 45-50 mol % non-cationic lipid.

In some aspects, the LNP of the disclosure comprises (a) 1-4 mol % PEG Lipid (e.g, PEG2000-C-DMA, PEG2000-S, or PEG750-C-DLA); (b) 50-60 mol % ionizable lipid (13-B43 or 18-B6); (c) 30-35 mol % cholesterol; and (d) 6-12 mol % Distearoyl phosphatidylcholine (DSPC).

In some aspects, the size for LNPs are between about 50-200 nm in diameter. In some aspects, the LNP particle size ranges from about 50-150 nm, about 50-100 nm, about 50-120 nm, or about 50-90 nm.

In some aspects, the LNP disclosed herein is formulated with a mRNA construct that encodes one or more of an enzyme, a growth factor, a cytokine, a receptor, a receptor ligand, a therapeutic protein, a hormone, a membrane protein, a membrane-associated protein, and antigen, and an antibody.

In some aspects, the LNP disclosed herein is formulated with a mRNA construct disclosed herein, which encodes an enzyme. In some aspects, the mRNA construct encodes an enzyme selected from ornithine transcarbamylase (OTC), Erythropoictin (EPO), argininosuccinate lyase (ASL), or matrix metalloproteinase-8 (MMP-8). In some aspects, the mRNA construct encodes an enzyme selected from Erythropoietin (EPO) or argininosuccinate lyase (ASL). In some aspects, the mRNA construct encodes Erythropoietin (EPO), e.g., human EPO (hEPO). In some aspects, the mRNA construct encodes argininosuccinate lyase (ASL). In some aspects, the mRNA construct encodes matrix metalloproteinase-8 (MMP-8), e.g., human MMP-8 (hMMP-8). In some aspects, the mRNA construct does not encode ornithine transcarbamylase (OTC).

### LNP Preparation

Those of skill in the art will appreciate that the following description is for illustration purposes only. The processes of the present disclosure are applicable to a wide range of lipid nanoparticle types and sizes. Further particles include, micelles, lipid-nucleic acid particles, virosomes, and the like. Those of skill in the art will know of other lipid LNPs for which the processes and apparatus of the present disclosure will be suitable.

In one aspect, the present method of encapsulating a polynucleic acid construct of the disclosure provides a lipid solution such as a clinical grade lipid synthesized under Good Manufacturing Practice (GMP), which is thereafter solubilized in an organic solution (e.g., ethanol). Similarly, a therapeutic product, e.g., a therapeutic active agent such as nucleic acid or other agent, is prepared under GMP. Thereafter, a therapeutic agent solution (e.g., mRNA) containing a buffer (e.g., citrate or ethanol) is mixed with a lipid solution solubilized in a lower alkanol to form a liposomal formulation. In preferred aspects of the disclosure, the therapeutic agent is “passively entrapped” in the liposome substantially coincident with formation of the liposome. However, those of skill in the art will realize that the processes and apparatus of the present disclosure are equally applicable to active entrapment or loading of the liposomes after formation of the LNP.

According to the processes and apparatus of the present disclosure, the action of continuously introducing lipid and buffer solutions into a mixing environment, such as in a mixing chamber, causes a continuous dilution of the lipid solution with the buffer solution, thereby producing a liposome substantially instantaneously upon mixing. As used herein, the phrase “continuously diluting a lipid solution with a buffer solution” (and variations) generally means that the lipid solution is diluted sufficiently rapidly in a hydration process with sufficient force to effectuate LNP generation. By mixing the aqueous solution with the organic lipid solution, the organic lipid solution undergoes a continuous stepwise dilution in the presence of the buffer (aqueous) solution to produce a liposome.

After the solutions, e.g., lipid solution and aqueous therapeutic agent (e.g., polynucleotide construct) solution, have been prepared, they are mixed together using, for example, a peristaltic pump mixer. In one aspect, the solutions are pumped at substantially equal flow rates into a mixing environment. In certain aspects, the mixing environment includes a “T”-connector or mixing chamber. In this instance, it is preferred that the fluid lines, and hence fluid flows, meet in a narrow aperture within the “T”-connector as opposing flows at approximately 180° relative to each other. Other relative introduction angles can be used, such as for example between 27° and 90° and between 90° and 180°. Upon meeting and mixing of the solution flows in the mixing environment, lipid LNPs are substantially instantaneously formed. Lipid LNPs are formed when an organic solution including dissolved lipid and an aqueous solution (e.g., buffer) are simultaneously and continuously mixed. Advantageously, and surprisingly, by mixing the aqueous solution with the organic lipid solution, the organic lipid solution undergoes a continuous stepwise dilution to substantially instantaneously produce a liposome. The pump mechanism can be configured to provide equivalent or different flow rates of the lipid and aqueous solutions into the mixing environment which creates lipid LNPs in a high alkanol environment.

Advantageously, the processes and apparatus for mixing of the lipid solution and the aqueous solution as provided herein provides for encapsulation of therapeutic agent in the formed liposome substantially coincident with liposome formation with an encapsulation efficiency of at least 90-95%. Further processing steps as discussed herein can be used to target a specific mRNA concentration by concentrating or diluting the sample, if desired.

In some aspects, the LNPs are formed having a mean diameter of less than about 150 nm (e.g., about 50-90 nm), which do not require further size reduction by high-energy processes such as membrane extrusion, sonication or microfluidization.

In certain aspects, LNPs form when lipids dissolved in an organic solvent (e.g., ethanol) are diluted in a stepwise manner by mixing with an aqueous solution (e.g., buffer). This controlled stepwise dilution is achieved by mixing the aqueous and lipid streams together in an aperture, such as a T-connector. The resultant lipid, solvent and solute concentrations can be kept constant throughout the LNP formation process.

In one aspect, using the processes of the present disclosure, a LNP is prepared by a two-stage step-wise dilution without gradients. For example, in the first stepwise dilution, LNPs are formed in a high alkanol (e.g., ethanol) environment (e.g., about 30% to about 50% v/v ethanol). These LNPs can then be stabilized by lowering the alkanol (e.g., ethanol) concentration to less than or equal to about 25% v/v, such as about 17% v/v to about 25% v/v, in a stepwise manner. In preferred aspects, with therapeutic agent present in the aqueous solution, or in the lipid solution, the therapeutic agent is encapsulated coincident with liposome formation.

In certain aspects, lipid stocks can be prepared in 100% ethanol, and then mixed with mRNA LNP in acetate buffer via a T-connector. The lipid and mRNA stocks can be mixed at a flow rate of 400 mL/min at the T-connector into a collection vessel containing PBS. In some aspects, lipids are initially dissolved in an alkanol environment of about 40% v/v to about 90% v/v, more preferably about 65% v/v to about 90% v/v, and most preferably about 80% v/v to about 90% v/v (A). Next, the lipid solution is diluted stepwise by mixing with an aqueous solution resulting in the formation of LNPs at an alkanol (e.g., ethanol) concentration of between about 37.5-50% (B). By mixing the aqueous solution with the organic lipid solution, the organic lipid solution undergoes a continuous stepwise dilution to produce a liposome. Further, lipid LNPs can be further stabilized by an additional stepwise dilution of the LNPs to an alkanol concentration of less than or equal to about 25%, preferably between about 15-25% (C).

In some aspects, for both stepwise dilutions (A→B and B→C), the resulting ethanol, lipid and solute concentrations are kept at constant levels in the receiving vessel. At these higher ethanol concentrations following the initial mixing step, the rearrangement of lipid monomers into bilayers proceeds in a more orderly fashion compared to LNPs that are formed by dilution at lower ethanol concentrations. Without being bound by any particular theory, it is believed that these higher ethanol concentrations promote the association of nucleic acid with cationic lipids in the bilayers. In certain aspects, the nucleic acid encapsulation occurs within a range of alkanol (e.g., ethanol) concentrations above 22%.

In certain aspects, after the lipid LNPs are formed, they are collected in another vessel, for example, a stainless steel vessel. In one aspect, a second dilution can be performed, e.g., at a rate of about 100-200 mL/min.

In one aspect, after the mixing step, the lipid concentration is about 1-10 mg/ml (e.g., about 7 mg/mL) and the therapeutic agent (e.g., mRNA) concentration is about 0.1-4 mg/mL.

After the mixing step, the degree of therapeutic agent (e.g., nucleic acid) encapsulation can be enhanced if the lipid LNP suspension is optionally diluted. For example, prior to dilution step, if the therapeutic agent entrapment is at about 30-40%, it can be increased to about 70-80% following incubation after the dilution step. In step, the liposome formulation is diluted to about 10% to about 40%, preferably about 20% alkanol, by mixing with an aqueous solution such as a buffer (e.g., PBS). Such further dilution is preferably accomplished with a buffer. In certain aspects, such further diluting the liposome solution is a continuous stepwise dilution. The diluted sample is then optionally allowed to incubate at room temperature.

After the optional dilution step, about 70-80% or more of the therapeutic agent (e.g., nucleic acid) is entrapped within the lipid LNP. In certain aspects, anion exchange chromatography is used.

In certain instances, the liposome solution is optionally concentrated about 2-6 fold, preferably about 4 fold, using for example, ultrafiltration (e.g., tangential flow dialysis). In one aspect, the sample is transferred to a feed reservoir of an ultrafiltration system and the buffer is removed. The buffer can be removed using various processes, such as by ultrafiltration.

In some aspects, the concentrated formulation is then diafiltrated to remove the alkanol. The alkanol concentration at the completion of step is less than about 1%. Preferably, lipid and therapeutic agent (e.g., nucleic acid) concentrations remain unchanged and the level of therapeutic agent entrapment also remains constant.

After the alkanol has been removed, the aqueous solution (e.g., buffer) is then replaced by dialfiltration against another buffer. Preferably, the ratio of concentrations of lipid to therapeutic agent (e.g., nucleic acid) remain unchanged and the level of nucleic acid entrapment is about constant. In certain instances, sample yield can be improved by rinsing the cartridge with buffer at about 10% volume of the concentrated sample. In certain aspects, this rinse is then added to the concentrated sample.

In certain aspects, sterile filtration of the sample can optionally be performed. In certain aspects, filtration is conducted at pressures below about 40 psi, using a capsule filter and a pressurized dispensing vessel with a heating jacket. Heating the sample slightly can improve the ease of filtration.

The sterile fill step can be performed using a processes for conventional liposomal formulations. In some aspects, the processes of the present disclosure results in about 50-60% of the input therapeutic agent (e.g., nucleic acid) in the final product. In certain preferred aspects, the therapeutic agent to lipid ratio of the final product is approximately 0.04 to 0.07.

Preparation of encapsulated LNPs can then be filtered under sterile conditions, aliquoted, and stored at −80° ° C.

### Copolymers

In some aspects, the composition of the disclosure further comprises a copolymer. In some aspects, the copolymer disclosed herein is a “membrane destabilizing polymers” or “membrane disruptive polymers.” Membrane destabilizing polymers or membrane disruptive polymers can directly or indirectly elicit a change, such as a permeability change for example, in a cellular membrane structure, such as an endosomal membrane for example, so as to permit an agent, for example an oligonucleotide or copolymer or both, to pass through such membrane structure. In some aspects, the membrane disruptive polymer can directly or indirectly elicit lysis of a cellular vesicle or otherwise disrupt a cellular membrane for example as observed for a substantial fraction of a population of cellular membranes.

The delivery agents, copolymers and compositions as disclosed herein can be useful in methods for the intracellular delivery of the polynucleotide constructs of the disclosure, to target cells, including target cells in vitro, ex vivo, and in vivo. In some aspects, a method of delivering a polynucleotide constructs, e.g., comprising an mRNA, to a target cell includes delivery to the cytosol of the cell.

### Compositions

The delivery agents disclosed herein can effectively transport polynucleotide constructs into cells both in vitro and in vivo. In some aspects, the polynucleotide construct of the disclosure is formulated with a delivery agent, e.g., an LNP. In some aspects, the compositions further comprises a pharmaceutically acceptable carrier.

Certain aspects of the disclosure are directed to a composition or method for increasing the amount of the protein of interest in a cell. In some aspects, the polynucleotide construct comprising a nucleic acid sequence comprising a codon optimized mRNA sequence comprising an open reading frame (ORF) encoding a functional protein of interest is formulated with an LNP and/or a copolymer into a composition. An protein of interest-encoding mRNA for formulation in the present disclosure typically further includes a poly(A) at its 3′ end (e.g., a polyA tail of greater than 80, e.g., 100 to 500 adenine residues), which can be added to a construct using well-known genetic engineering techniques (e.g., via PCR or enzymatic Poly-A tail). In some aspects, the poly(A) is between 100 and 500 nucleotides in length.

### Methods of Use

Certain aspects of the disclosure are directed to increasing the amount of a protein of interest in a cell by contacting the cell with a composition comprising a polynucleotide construct disclosed herein and a pharmaceutically acceptable diluent or carrier. In some aspects, the polynucleotide construct is formulated with an LNP disclosed herein. In further aspects, the polynucleotide can be formulated with a copolymer.

Some aspects are directed to a method for increasing the expression of a protein of interest in a cell comprising administering to the cell a composition comprising the polynucleotide construct of the disclosure. The cell can be any cell. Examples of cells that can be used include, but are not limited to, liver, heart, lung, brain, kidney, stomach, breast, muscle, gallbladder, spleen, bone marrow, pancreas, bladder, eye, large intestine, small intestine, nose, ovary, parathyroid gland, pituitary gland, adrenal gland, prostate, salivary gland, skin, hair, and thymus gland cells.

A method for treating a disease or disorder comprising administering to a subject in need thereof a therapeutically effective amount of a composition comprising the polynucleotide construct of the disclosure. The disease or disorder can be any disease or disorder.

Other aspects of the disclosure are directed to the use of a polynucleotide constructs of the disclosure or composition of the disclosure, or a vector of the disclosure, or a host cell of the disclosure, for the manufacture of a medicament for the treatment of a disease or disorder in a subject in need thereof. The disease or disorder can be any disease or disorder.

A disease or condition associated with defective gene expression and/or activity in a subject treatable by the methods disclosed herein. In some aspects, the constructs, polynucleotides, and/or compositions of the disclosure can be suitable for use in gene therapy. In some aspects, the combination of construct elements (e.g., Cap, 5′UTR, 3′UTR, and polyA) provides for mRNA that are have improved stability, expression, and/or efficacy. In some aspects, the administration of the mRNA constructs of the disclosure with an LNP provides improved stability, expression, and/or efficacy.

In certain aspects, the disease or condition associated with defective gene expression is a disease characterized by a deficiency in a functional polypeptide (also referred to herein as a “disease associated with a protein deficiency”). A delivery agent, e.g., LNP, of the disclosure can be formulated into a composition comprising a messenger RNA (mRNA) molecule encoding a protein corresponding to a genetic defect that results in a deficiency of the protein. For treatment of the disease associated with the protein deficiency, the polynucleic acid construct, e.g., comprising an mRNA, formulation can be administered to a subject (e.g., mammal such as, for example, a mouse, non-human primate, or human) for delivery of the mRNA to an appropriate target tissue, where the mRNA is translated during protein synthesis and the encoded protein is produced in an amount sufficient to treat the disease.

In some aspects, the disease is associated with a deficiency in a protein selected from an enzyme, a growth factor, a cytokine, a receptor, a receptor ligand, a hormone, a membrane protein, or a membrane-associated protein.

In some aspects, the protein of interest an enzyme. In some aspects, the protein of interest is an enzyme selected from ornithine transcarbamylase (OTC), Erythropoietin (EPO), argininosuccinate lyase (ASL), or matrix metalloproteinase-8 (MMP-8). In some aspects, the protein of interest is an enzyme selected from Erythropoietin (EPO) or argininosuccinate lyase (ASL). In some aspects, the protein of interest is Erythropoietin (EPO), e.g., human EPO (hEPO). In some aspects, the protein of interest is argininosuccinate lyase (ASL). In some aspects, the protein of interest is matrix metalloproteinase-8 (MMP-8), e.g., human MMP-8 (hMMP-8). In some aspects, the protein of interest is not ornithine transcarbamylase (OTC).

In some aspects, the disease to be treated is an infectious disease or a cancer. In some aspects, the disease is treated with a genetic vaccine encoding an antibody or antigen.

In some aspects, the protein of interest is an antigen, such as a SARS COV2 protein, e.g., SARS-COV2 spike protein, or an influenza antigen, e.g., Hemagglutinin (HA).

An example of a method of treating a disease or condition associated with defective gene expression, infection, and/or activity in a subject, such as a mammal for example, includes administering to a mammal in need thereof a therapeutically effective amount of a polynucleotide construct comprising a nucleic acid sequence comprising a codon optimized mRNA sequence comprising an open reading frame (ORF) encoding a functional protein of interest is formulated with an LNP and/or a copolymer into a composition.

In some aspects, an protein of interest-encoding mRNA for formulation in the present disclosure includes a poly(A) at its 3′ end (e.g., a polyA tail of greater than 80, e.g., 100 to 500 adenine residues).

A further example of a method for treating a disease or condition associated with defective gene expression includes a method of treating a subject having a deficiency in a functional polypeptide comprising administering to the subject a composition comprising at least one mRNA molecule at least a portion of which encodes the functional polypeptide where following administration the expression of the functional polypeptide is greater than before administration.

The efficacy of an mRNA composition for treating a disease can be evaluated in vivo in animal models of disease.

In certain aspects, the polynucleotide constructs and compositions of the present disclosure is useful in the preparation of a medicament for the treatment of a disease or condition associated with defective gene expression and/or activity in a subject.

In some aspects, the defective gene encodes an enzyme, e.g., Erythropoietin (EPO). In some aspect, the mRNA constructs and compositions of the present disclosure encode Erythropoietin (EPO) for treatment of anemia, e.g., due to a chronic kidney disease or disorder.

In some aspects, the defective gene encodes an enzyme, e.g., argininosuccinate lyase (ASL). In some aspect, the mRNA constructs and compositions of the present disclosure encode argininosuccinate lyase (ASL) for treatment of ASL deficiency.

In some aspects, the defective gene encodes an enzyme, e.g., matrix metalloproteinase-8 (MMP-8). In some aspect, the mRNA constructs and compositions of the present disclosure encode matrix metalloproteinase-8 (MMP-8) for treatment of a MMP-8 deficiency.

In some aspects, the defective gene is an enzyme, e.g., ornithine transcarbamylase (OTC). In some aspect, the mRNA constructs and compositions of the present disclosure encode ornithine transcarbamylase (OTC) for treatment of OTC deficiency.

In some aspects, the mRNA constructs and compositions of the present disclosure encode an antigen, e.g., Hemagglutinin (HA) or a SARS-COV2 protein (e.g., a SARS-CoV2 spike protein). In some aspect, the mRNA constructs and compositions (e.g., a vaccine) of the present disclosure encode an antigen for treatment or prevention of influenza or a COVID infection.

The polynucleotide constructs and compositions of the present disclosure can be administered in a variety of routes of administration such as parenteral, oral, topical, rectal, inhalation and the like. Formulations will vary according to the route of administration selected. In some aspects, the route of administration is intravenously, intramuscularly, intracutaneously, subcutaneously, intraduodenally, or intraperitoneally.

Determination of the proper dosage for a particular situation is within the skill of the art. Effective doses of the compositions of the present disclosure vary depending upon many different factors, including means of administration, target site, physiological state of the patient, whether the patient is human or an animal, other medications administered, as well as the specific activity of the composition itself and its ability to elicit the desired response in the individual. Usually, the patient is a human, but in some diseases, the patient can be a nonhuman mammal.

### EXAMPLES

### Example 1. Preparation of Polynucleotide Constructs Encoding an Exemplary Protein of

### Interest

An OTC polynucleotide constructs were prepared by In Vitro Transcription (IVT) using a plasmid DNA construct. The plasmid DNA construct contained the instructions for the 5′UTR, ORF and 3′UTR while the chemical modification (e.g. Pseudouridine) was determined by the addition of the desired nucleotide to the IVT reaction. To start, the plasmid DNA was linearized using 5 units of Xbal restriction enzyme per ug of plasmid DNA. After an overnight incubation at 37 degrees the DNA was purified by phenol/chloroform extraction. An IVT reaction in addition to co-transcriptional capping (e.g., Cap1) was performed for 3 hours at 37 degrees using T7 Polymerase and CleanCap. After the IVT reaction, the resultant mRNA product was purified via DNase treatment followed by Diafiltration. The purified mRNA was then enzymatically Poly adenylated with 300 units of Poly A polymerase per mg RNA and incubated for between 15 and 60 minutes, depending on the desired Poly A tail length. The mRNA product was then purified by Diafiltration and HPLC before being adjusted to a desired concentration, sterile filtered and aliquoted.

### Example 2. Effects of Poly(A) Tail Length on Potency and Tolerability

OTC mRNA constructs as described in Example 1 were prepared with a poly(A) tails having variable lengths. In a first experiment, OTC mRNA was transcribed and the crude transcript was used as a template for a reaction with pre-warmed or cold PolyA polymerase. In a second experiment, OTC mRNA was transcribed, purified, and the purified transcript was used as a template for a reaction with pre-warmed or cold PolyA polymerase. In a third experiment, the reaction time to yield the correct PolyA tail length was determined.

PolyA experiments 1 and 2 resulted in no significant difference in the length of PolyA tails generated. Additionally, enzyme temperature did not affect run performance. In experiments 1 and 2, the reaction time was 30 min. In experiment 3, reaction times of 45, 60, and 75 min were tested. 60 and 75 minute reaction times were able to generate PolyA tails over 300 nucleotides (nts) in length. Although the longer reaction times produced longer tails, the reaction time also impacted the purity of the product.

To assess the effect of different poly(A) tail length (encoded or enzymatic) on potency and tolerability, a rat repeated dose study was performed. An OTC construct comprising mRNA with different poly(A) tail lengths (80, 161, 208, 262, 322, or 440 nts) encapsulated in LNP2 (PEG2000-S:13-B43:Cholesterol:DSPC) was administered to male Srague Dawley rats (7-8 weeks old) at DO, 7, and 14 (Table 2A). The experiment was terminated at D1 (24 h post-dose) or D15 (24 h post-last-dose). The Z-Avg, PDI, and % Encaps of each formulation administered is provided in Table 2B. All formulations were tested for endotoxin by in-house LAL assay. All formulations were below 2 EU/mL when at 0.5 mg/mL.

TABLE 2A
Administration and Dosing of LNP2 Formulations

| | | | | Dose | | |
|---|---|---|---|---|---|---|
| Group | N | LNP | Payload | (mg/kg) | Dose Day | Terminal |
| 1a/1b | 4 | PBS | — | — | D 0 (n = 2) | N = 2 on D 1 (24 h) |
| | | | | | D 0, 7, 14 | N = 2 on D 15 (24 h) |
| | | | | | (n = 2) | |
| 2 | 4 | LNP2 | OTC mRNA | 0.25 | D 0 | D 1 (24 h post-dose) |
| 3 | 4 | (13- | Construct - 80 | | D 0, 7, 14 | D 15 (post-last dose) |
| | | B43) | nts Poly(A) | | | |
| | | | encoded | | | |
| 4 | 4 | | OTC mRNA | | D 0 | D 1 (24 h post-dose) |
| 5 | 4 | | Construct - 161 | | D 0, 7, 14 | D 15 (post-last dose) |
| | | | nts Poly(A) | | | |
| 6 | 4 | | OTC mRNA | | D 0 | D 1 (24 h post-dose) |
| 7 | 4 | | Construct - 208 | | D 0, 7, 14 | D 15 (post-last dose) |
| | | | nts Poly(A) | | | |
| 8 | 4 | | OTC mRNA | | D 0 | D 1 (24 h post-dose) |
| 9 | 4 | | Construct - 262 | | D 0, 7, 14 | D 15 (post-last dose) |
| | | | nts Poly(A) | | | |
| 10 | 4 | | OTC mRNA | | D 0 | D 1 (24 h post-dose) |
| 11 | 4 | | Construct - 322 | | D 0, 7, 14 | D 15 (post-last dose) |
| | | | nts Poly(A) | | | |
| 12 | 4 | | OTC mRNA | | D 0 | D 1 (24 h post-dose) |
| 13 | 4 | | Construct - 440 | | D 0, 7, 14 | D 15 (post-last dose) |
| | | | nts Poly(A) | | | |

TABLE 2B
LNP2 Formulation Characteristics

| Formulation | Poly(A) Length | Z-Avg (nm) | PDI | % Encapsulation |
|---|---|---|---|---|
| LNP2 (13- | 80 | 86 | 0.08 | 96 |
| B43) | 208 | 76 | 0.08 | 97 |
| | 161 | 76 | 0.06 | 97 |
| | 262 | 79 | 0.06 | 95 |
| | 322 | 83 | 0.04 | 97 |
| | 440 | 79 | 0.05 | 96 |

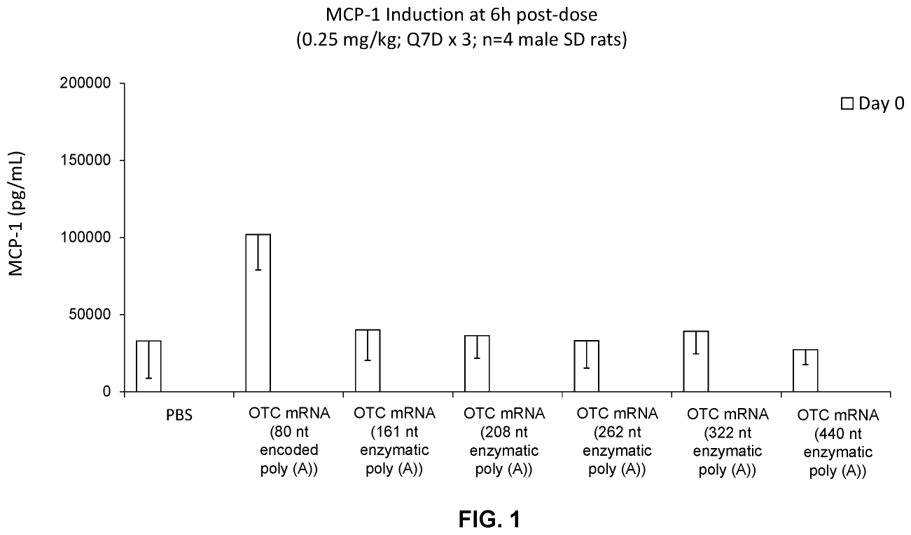

Monocyte Chemoattractant Protein-1 (MCP-1) induction levels 6 h after the first dose were analyzed for various polyA constructs, and the results are shown in FIG. 1.

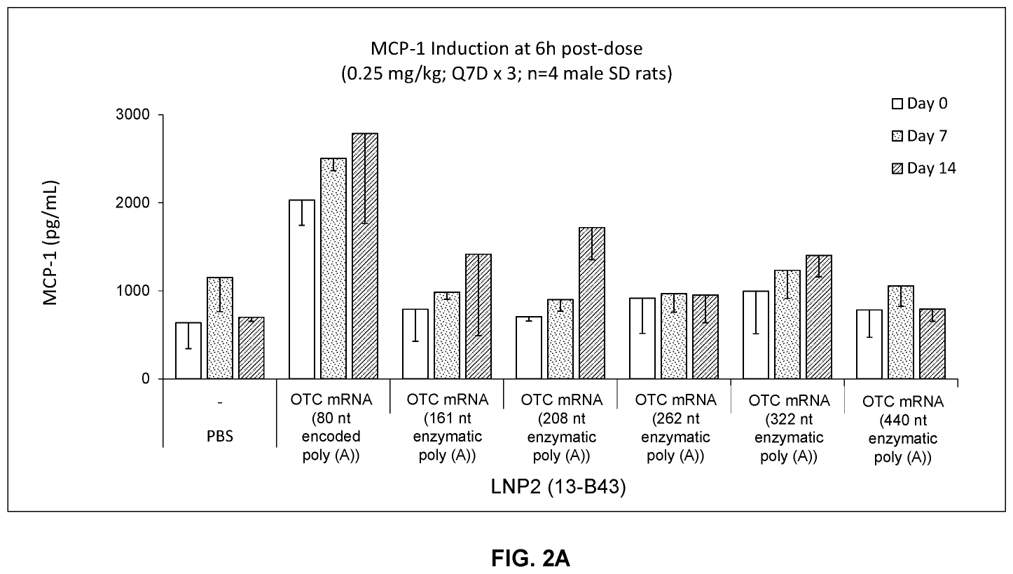

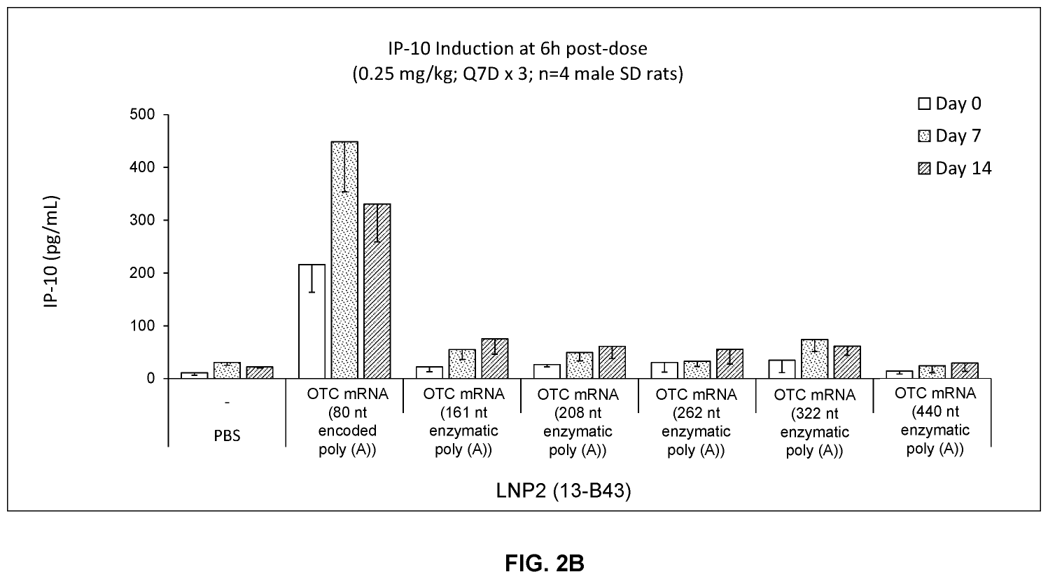

To analyze the induction of immune responses to administration of the LNPs formulated with OTC constructs including mRNA with various polyA tail lengths upon repeat dosing, tail pokes were obtained 6 h after dosing on each dosing day and rat cytokine induction was quantified. Monocyte Chemoattractant Protein-1 (MCP-1) induction levels 6 h after dosing (Day 0, Day 7, and 14) were analyzed (FIG. 2A). The OTC mRNA having a 80 nts encoded Poly(A) resulted in higher MCP-1 induction levels compared to OTC mRNA constructs having 161, 208, 262, 322, or 440 nt enzymatic Poly(A) tails. MCP-1 and interferon γ-induced protein 10 (IP-10) induction levels were analyzed at 6 h post-dosing on days D0, D7, and D14 (FIG. 2B). All responses were compared to PBS control group. The OTC mRNA construct with 80 nt encoded Poly(A) tail showed higher MCP-1 (FIG. 2A) and IP-10 (FIG. 2B) induction compared to the tested OTC mRNA constructs with enzymatic Poly(A) tails greater than 80 nucleotides.

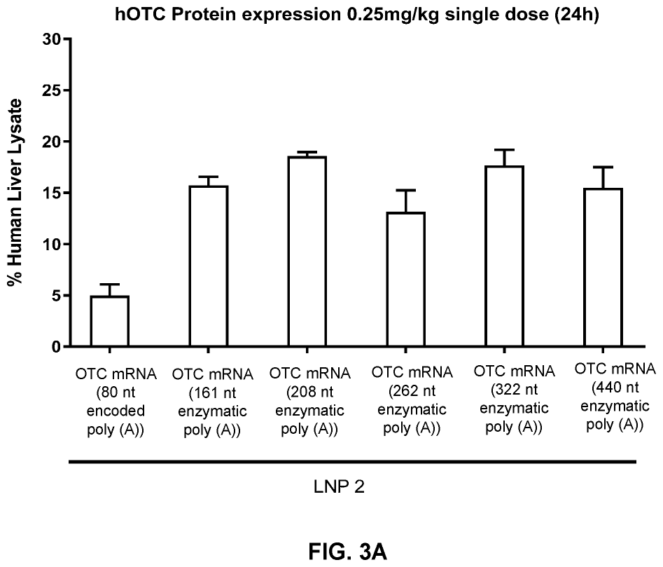

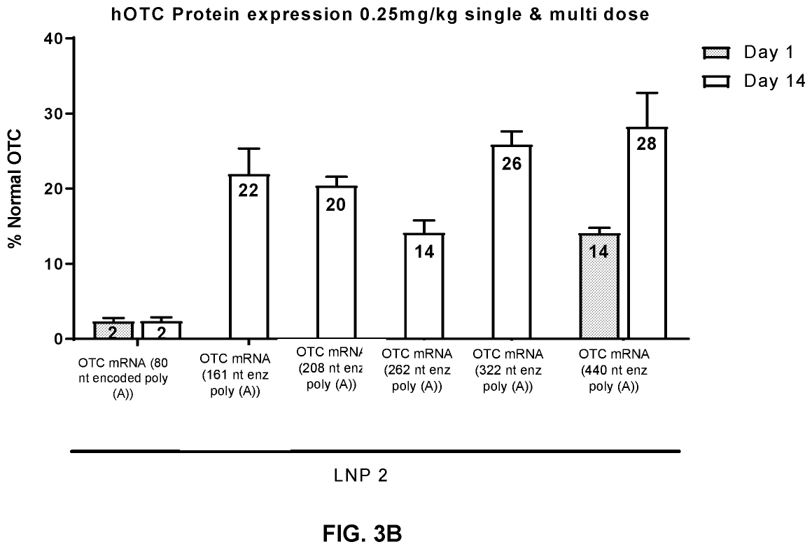

To analyze OTC protein expression, rat liver samples were obtained 24 hr post-last-dose and flash frozen. The OTC construct having the 80 nucleotide encoded Poly(A) had the lowest hOTC protein expression in the liver compared the OTC constructs having the enzymatic Poly(A) tails greater than 80 nucleotides (FIG. 3A and FIG. 3B).

### Example 3. Modified OTC mRNA Constructs

To assess the effect of chemical modifications on potency and tolerability, a mouse study was performed. OTC mRNA prepared in Example 1 (having a polyA tail range ˜180-480 nucleotides long) was chemically modified with either pseudouridine (PsU), N1-methyl-pseudouridine (N1MePsU), or 5-methoxyduridine (5MoU) (Table 3A) using TriLink methods.

The chemically modified mRNA was formulated into either LNP1 or LNP2 (PEG2000-S:13-B43:Cholesterol:DSPC) (Table 3B) and administered to mice (0.5 mg/kg) (Table 3C).

TABLE 3A
Chemical Modification of mRNAs

| Group | N | Composition | mRNA & Mods | Dose | |
|---|---|---|---|---|---|
| 1 | 4 | PBS | | 10 | mL/kg |
| 2 | 4 | LNP1 | OTC mRNA-PsU | 0.5 | mg/kg |

| | | | |
|---|---|---|---|
| 3 | 4 | LNP2 (13-B43) | |
| 4 | 4 | LNP1 | OTC mRNA- |
| 5 | 4 | LNP2 (13-B43) | N1MePsU |
| 6 | 4 | LNP1 | OTC mRNA-5MoU |
| 7 | 4 | LNP2 (13-B43) | |

TABLE 3B
LNP Formulation of Chemically Modified mRNAs

| | | | mRNA | | | |
|---|---|---|---|---|---|---|
| | | | length | Poly(A) | Expected | |
| | | | before | tail | Total | |
| | | Purity | PAP | length | length | ~ Actual |
| Variant | Purification | (%) | (nt) | (nt) | (nt) | length (CE) |
| PsU | HPLC | 93 | 1226 | 322 | 1548 | 1600 |
| 5MOU | Silica | 85 | 1226 | 186 | 1412 | 1700 |
| N1MePsU | HPLC | 90 | 1226 | 470 | 1696 | 1800 |

TABLE 3C
Administration of Chemically Modified mRNA

| | | Z-Avg | | Encaps | Conc |
|---|---|---|---|---|---|
| LNP | Payload | (nm) | PDI | (%) | (mg/mL) |
| LNP1 | OTC mRNA- | 78 | 0.09 | 96 | 0.045 |
| LNP2 | PsU | 84 | 0.12 | 96 | 0.045 |
| (13-B43) | | | | | |
| LNP1 | OTC mRNA- | 73 | 0.06 | 96 | 0.048 |
| LNP2 | N1MePsU | 77 | 0.09 | 96 | 0.048 |
| (13-B43) | | | | | |
| LNP1 | OTC mRNA- | 73 | 0.07 | 96 | 0.048 |
| LNP2 | 5MoU | 77 | 0.09 | 95 | 0.050 |
| (13-B43) | | | | | |

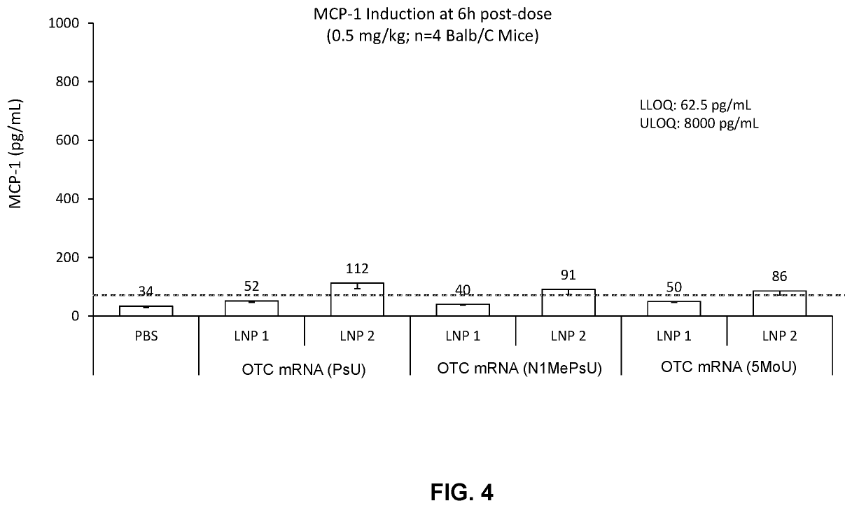

MCP-1 levels were analyzed after administration of the modified OTC mRNA formulations (FIG. 4). There were no significant differences in MCP-1 response between the different tested OTC mRNA chemical modifications. LNP2 (PEG2000-S:13-B43:Cholesterol:DSPC) was slightly more stimulatory compared to LNP1.

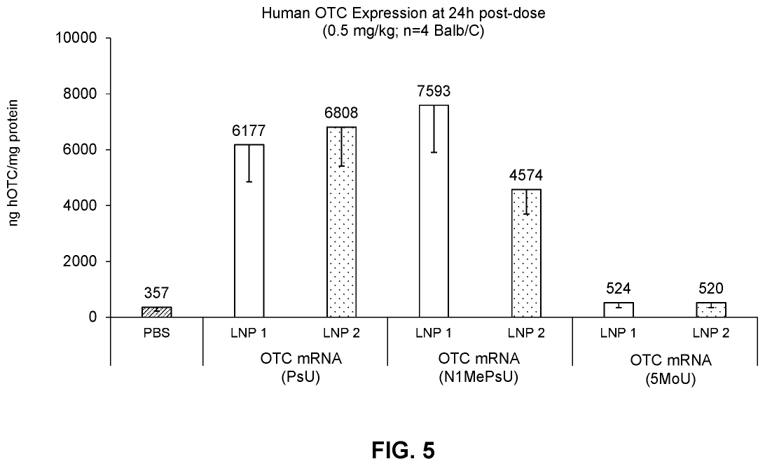

Next, human OTC expression was analyzed by ELISA (FIG. 5). There were similar levels of OTC expression between OTC mRNA PsU and N1MePsU modifications in both LNPs. The lowest OTC expression was detected in OTC mRNA 5MoU-LNP treated animals. OTC mRNA N1MePsU-LNP1 treated animals had higher OTC expression than OTC mRNA PsU-LNP1 treated animals. OTC mRNA PsU-LNP2 treated animals had higher OTC expression than OTC mRNA N1MePsU treated animals.

### Example 4. OTC mRNA-LNP Tolerability and OTC Expression in Rats

OTC mRNA-PsU potency and tolerability was evaluated in a rat repeat dose study. OTC mRNA-PsU (0.25 mg/kg) was formulated in either LNP1 (PEG2000-C-DMA:13-B43:Cholesterol:DSPC), LNP2 (PEG2000-S:13-B43:Cholesterol:DSPC or PEG2000-S:18-B6:Cholesterol:DSPC), or LNP3 (PEG750-C-DLA:18-B6:Cholesterol:DSPC) and administered to mice on Day 0, 7, and 14 (Table 4A). EPO and LUC were carried in LNP1 and administered as controls.

TABLE 4A
Administration and Dosing of OTC mRNA Construct-PsU

| | | | | Dose | Dose | |
|---|---|---|---|---|---|---|
| Group | N | LNP | Payload | (mg/kg) | Day | Terminal |
| 1 | 2 | PBS | — | — | D 0 | D 1 (24 h) |
| 2 | 3 | LNP1 | EPO | 0.25 | D 0, | D 14 (24 h) |
| | | | (5MoU) | | 7, 14 | |
| 3 | 3 | | LUC | | D 0, | D 14 (24 h) |
| | | | (5MoU) | | 7, 14 | |
| 4 | 3 | | OTC | | D 0 | D 1 (24 h |
| | | | mRNA | | | post-last |
| | | | (262 nts | | | dose) |
| | | | Poly(A)) | | | |
| | | | (PsU) | | | |
| 5 | 3 | | OTC | | D 0, | D 15 (24 h |
| | | | mRNA | | 7, 14 | post-last |
| | | | (262 nts | | | dose) |
| | | | Poly(A)) | | | |
| | | | (PsU) | | | |
| 6 | 3 | LNP2 (13- | OTC | | D 0 | D 1 (24 h |
| | | B43) | mRNA(262 | | | post-last |
| | | | nts | | | dose) |
| 7 | 3 | | Poly(A)) | | D 0, | D 15 (24 h |
| | | | (PsU) | | 7, 14 | post-last |
| | | | | | | dose) |
| 8 | 3 | LNP2 (18- | | | D 0 | D 1 (24 h |
| | | B6) | | | | post-last |
| | | | | | | dose) |
| 9 | 3 | | | | D 0, | D 15 (24 h |
| | | | | | 7, 14 | post-last |
| | | | | | | dose) |
| 10 | 3 | LNP3 | | | D 0 | D 1 (24 h |
| | | | | | | post-last |
| | | | | | | dose) |
| 11 | 3 | | | | D 0, | D 15 (24 h |
| | | | | | 7, 14 | post-last |
| | | | | | | dose) |

The Z-Avg. PDI, and % Encaps of each formulation administered is provided in Table 4B. Input batch size was 3 mg. LNPs were formulated with 100 mM acetate. pH5 and worked up on TFU. Aliquots were stored at −80° C. and test articles were prepared on each day of dosing.

TABLE 4B
LNP1, LNP2, and LNP3 Formulation Characteristics

| LNP | | Z-avg | | Encaps | Recovery | |
|---|---|---|---|---|---|---|
| Composition | Payload | (nm) | PDI | (%) | (%) | Endotoxin |
| LNP1 | Human | 74 | 0.15 | 98 | 84 | <5 EU/mL |
| | EPO | | | | | |
| LNP1 | LUC | 71 | 0.10 | 96 | 74 | <5 EU/mL |
| LNP1 | OTC | 74 | 0.09 | 96 | 74 | <5 EU/mL |
| | mRNA | | | | | |
| | (262 nts | | | | | |
| | Poly(A)) | | | | | |
| LNP2 (13- | OTC | 74 | 0.11 | 96 | 76 | <5 EU/mL |
| B43) | mRNA | | | | | |
| | (262 nts | | | | | |
| | Poly(A)) | | | | | |
| LNP2 (18- | OTC | 71 | 0.10 | 94 | 78 | <5 EU/mL |
| B6) | mRNA | | | | | |
| | (262 nts | | | | | |
| | Poly(A)) | | | | | |
| LNP3 | OTC | 76 | 0.13 | 90 | 74 | <5 EU/mL |
| | mRNA | | | | | |
| | (262 nts | | | | | |
| | Poly(A)) | | | | | |

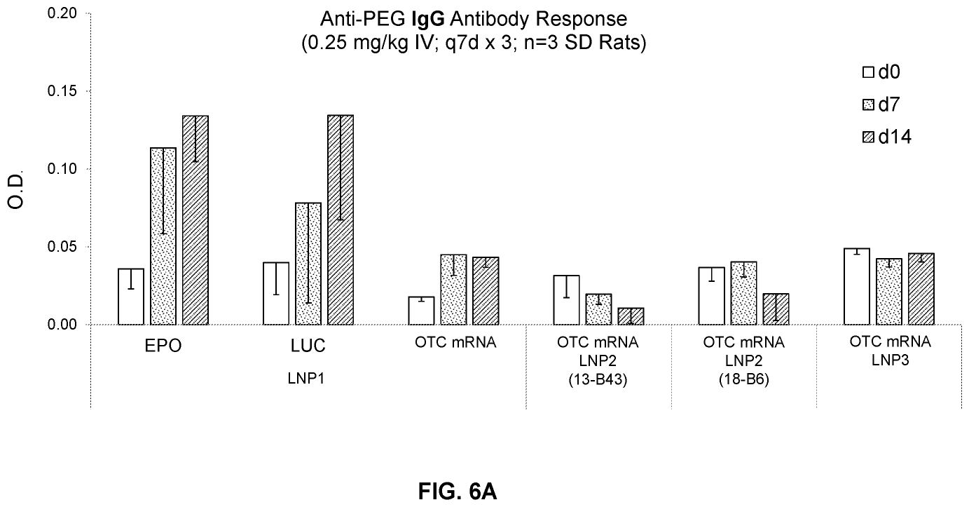

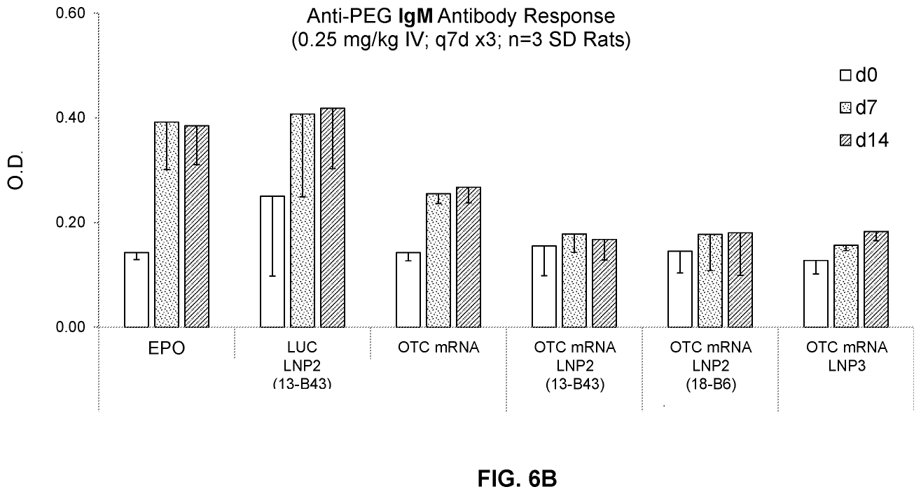

To examine PEG-antibody levels, blood was collected pre-dose on each dosing day (DO, 7, and 14). Both anti-PEG IgG (FIG. 6A) and anti-PEG IgM (FIG. 6B) antibody responses were quantified. Anti-PEG antibodies were observed in rats treated with LNP1 only. The tested OTC mRNA constructs were less immunogenic than the EPO and LUC payloads. Generation of anti-PEG antibodies with LNP1 resulted in accelerated blood clearance and loss of potency upon repeated dose (data not shown).

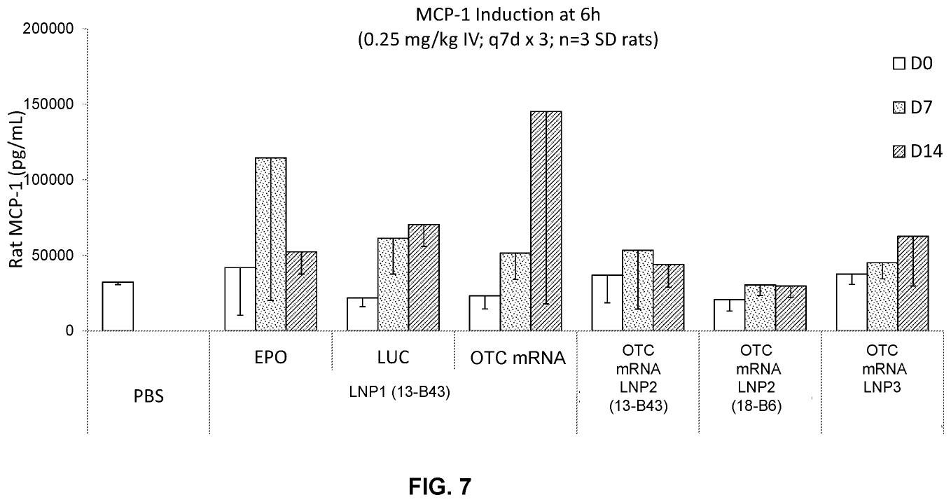

To examine MCP-1 induction, blood was collected 6 h after each dosing. There was little to no increase in MCP-1 upon repeat dose of LNP containing OTC mRNA constructs which correlates with lower immunogenicity (FIG. 7).

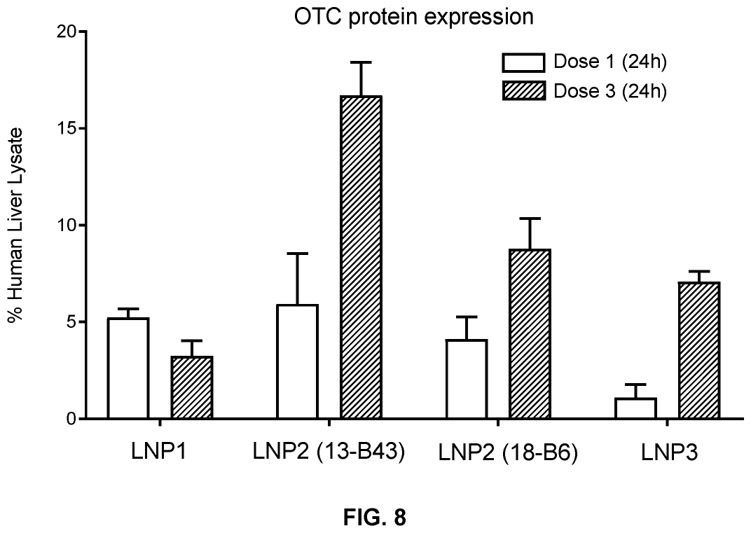

To examine OTC expression levels, blood was collected pre-dose on each dosing day. LNP2 formulations were the most potent, while LNP1 formulations were the least potent (FIG. 8). The highest accumulation of OTC protein was with LNP2 formulations. This data is supported with immunogenicity data which showed no antibodies were produced and there was no accelerated blood clearance. OTC mRNA constructs-LNP2 compositions also had lower repeat-dose MCP-1 levels.

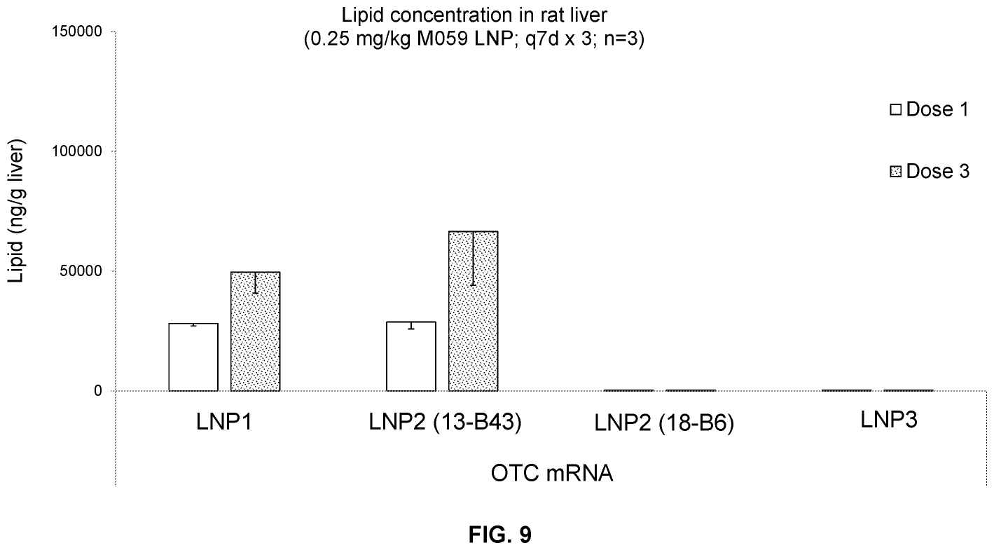

Lipid clearance was quantified 24 h post-dosing by mass spectroscopy. A single dose study showed that LNP1 and LNP2 (13-B43) were present at 14 days post-dose while LNP2 (18-B6) and LNP3 clearly rapidly by 6 h post-dose (data not shown). Repeat dose with OTC mRNA construct-LNP1 or OTC mRNA construct-LNP2 (13-B43) resulted in lipid accumulation in liver (FIG. 9). No accumulation of OTC mRNA construct-LNP2 (18-B6) or OTC mRNA constructs-LNP3 was seen, even upon repeated dose (all levels <LLOQ of 500 ng/g).

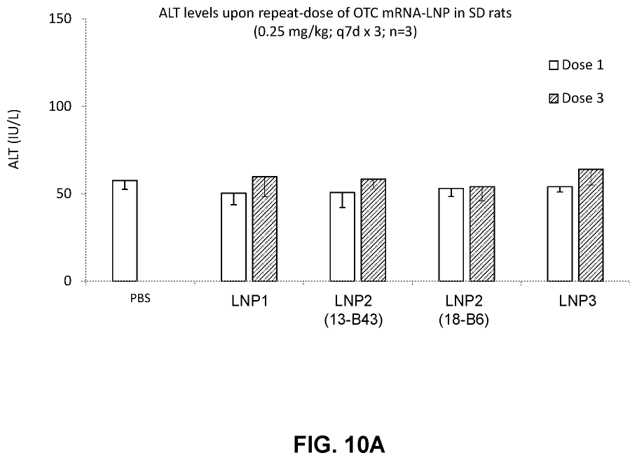

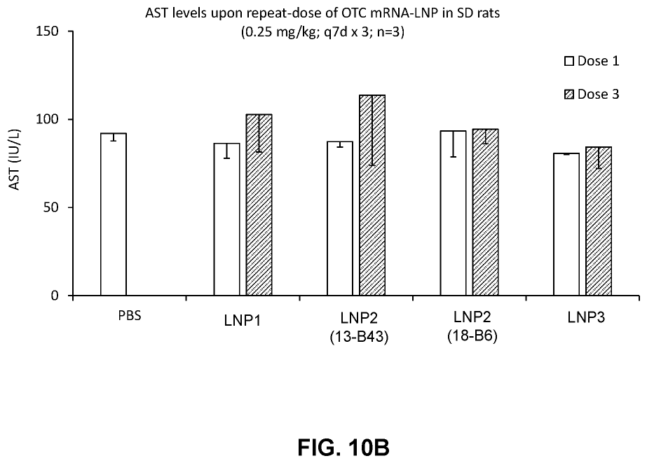

To analyze for markers of liver damage, alanine aminotransferase (ALT) and aspartate aminotransferase (AST) levels were quantified. Serum was collected at 24 h on the first and last day of dosing. There were no significant changes in ALT/AST levels upon repeat dose (0.25 mg/kg administered weekly×3 doses; 0.75 mg/kg total) (FIGS. 10A and 10B). Both LNP1 and LNP2 (13-B43) formulation groups have relatively higher AST compared to the LNP2 (18-B6) and LNP3 formulations after the third dose.

### Example 5. Single Vs. Repeat-Dose Lipid Clearance in Rats

Lipid-clearance following single and repeated-dose administration of OTC mRNA construct-LNP was evaluated. OTC mRNA was formulated in LNP2 (PEG2000-S:13-B43:Cholesterol:DSPC) and administered to rats at 0.25 mg/kg per dose. For single dose, rats were administered the formulation at DO and terminal time points were at 30 min, 1 h, 3 h, 6 h, and 24 h after administration (Table 5A). A high single dose (2 mg/kg) was administered at DO and the terminal time point was D1. For repeated dosing, rats were administered the formulation once every seven days for up to 49 days (day 7, 14, 21, 28, 35, 42, and 49). After the 8th treatment (Day 49) terminal time points were collected at 30 min, 1 h, 3 h, 6 h, and 24 h (Day 50) after dosing. PBS was administered as a control (5 ml/kg) at DO, 7, 14, 21, 28, 35, 42 and 49. The Z-Avg, PDI, and % Encaps of each formulation administered is provided in Table 5B.

TABLE 5A
Single and Repeated Dosing of OTC mRNA construct-LNP2

| | | | | | | Terminal | |
|---|---|---|---|---|---|---|---|
| Group | N | Test Article | Dose | | Dose Day | Time point | |
| 1 | 3 | PBS | 5 | mL/kg | D 0, 7, 14, 21, 28, 35, 42, 49 | 24 h | (D 50) |
| 2 | 3 | LNP2 (13- | 2 | mg/kg | D 0 (2 mg/kg total) (1.8 mg) | D | 1 |
| 3 | 3 | B43) | 0.25 | mg/kg | D 0 (0.25 mg/kg total) | 30 | min |

| | | | | |
|---|---|---|---|---|
| 4 | 3 | | 1 | h |
| 5 | 3 | | 3 | h |
| 6 | 3 | | 6 | h |
| 7 | 3 | | 24 h | (D 1) |
| 8 | 3 | D 0, 7 (0.5 mg/kg total) | D | 8 |
| | | (0.45 mg) | | |
| 9 | 3 | D 0, 7, 14 (0.75 mg/kg | D | 15 |
| | | total) (0.675 mg) | | |
| 10 | 3 | D 0, 7, 14, 21 (1 mg/kg | D | 22 |
| | | total) (0.9 mg) | | |
| 11 | 3 | D 0, 7, 14, 21, 28 | D | 29 |
| | | (1.25 mg/kg total) | | |
| | | (1.125 mg) | | |
| 12 | 3 | D 0, 7, 14, 21, 28, 35 | D | 36 |
| | | (1.5 mg/kg total) (1.35 mg) | | |
| 13 | 3 | D 0, 7, 14, 21, 18, 35, 42 | D | 43 |
| | | (1.75 mg/kg total) | | |
| | | (1.575 mg) | | |
| 14 | 3 | D 0, 7, 14, 21, 28, 35, 42, 49 | 30 | min |
| 15 | 3 | (2 mg/kg total) | 1 | h |
| 16 | 3 | | 3 | h |
| 17 | 3 | | 6 | h |
| 18 | 3 | | 24 hr | (D 50) |

TABLE 5B
OTC mRNA Construct-LNP2 (13-B43) Formulation Characteristics

| | | | | | | | Input | |
|---|---|---|---|---|---|---|---|---|
| LNP | | | Z-avg | | Encaps | NA | mRNA | Recovery |
| Composition | Payload | | (nm) | PDI | (%) | mg/mL | (mg) | (%) |
| LNP2 (13- | OTC | PF | 80 | 0.11 | 98 | 1.721 | 34.4 | 79 |
| B43) | mRNA | SF | 82 | 0.07 | 95 | | | |
| | construct | F/T | 82 | 0.07 | 96 | | | |

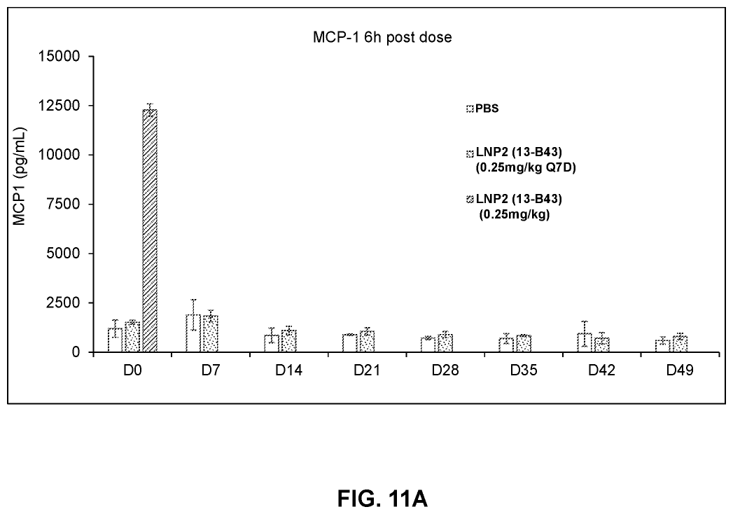

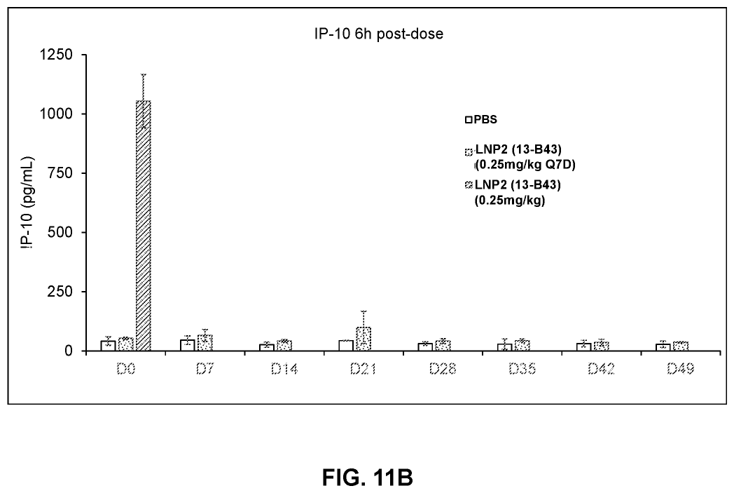

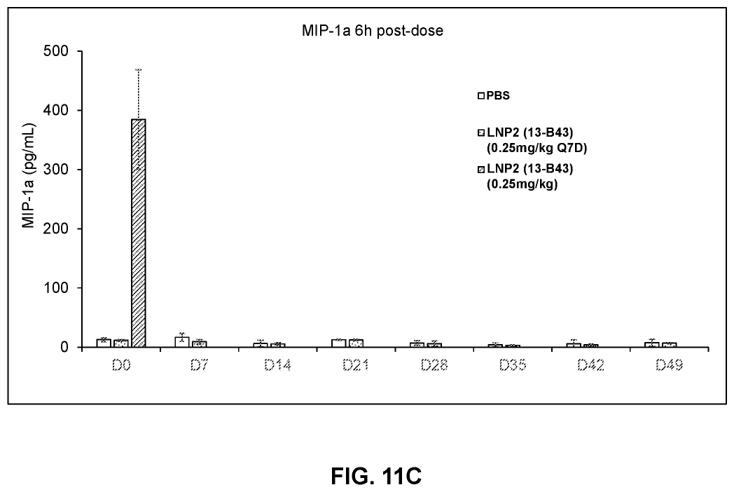

To measure cytokine response, blood was collected at all terminal time points. The cytokines measured were MCP-1, IP-10 and Macrophage inflammatory protein 1α (MIP-1α). There was no cytokine response generated from weekly repeated dose of 0.25 mg/kg (FIGS. 11A-11C). There was a significant cytokine response upon administration of a single dose at 2 mg/kg.

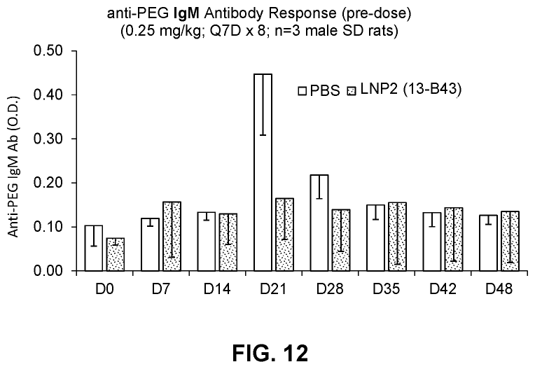

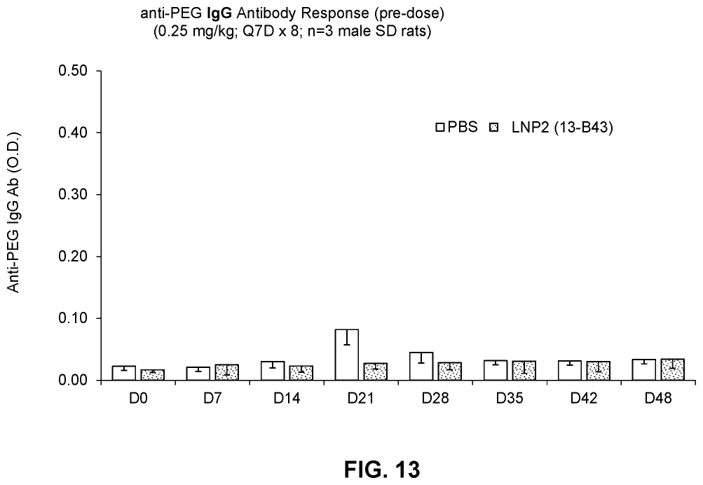

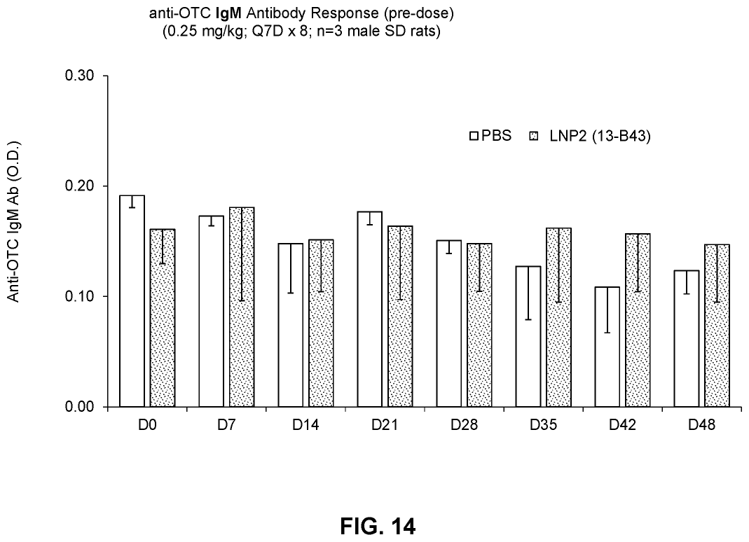

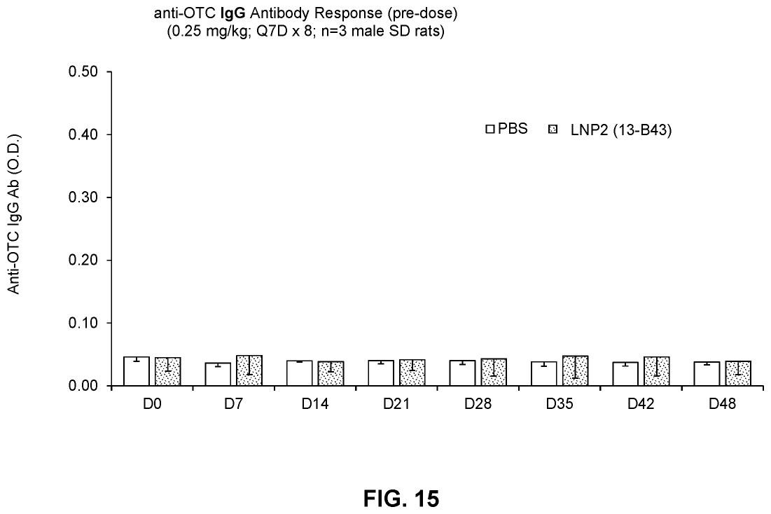

To examine PEG and OTC antibody levels, blood was collected prior to each dose. There was no trend towards increasing levels of anti-PEG IgM with repeated administration (FIG. 12). Similarly, there was no increase in anti-PEG IgG levels with repeated administration of OTC mRNA construct-LNP2 (FIG. 13). No anti-OTC IgM antibodies were detected with repeated administration (FIG. 14). Likewise, no anti-OTC IgG antibodies were detected with repeated administration of OTC mRNA construct-LNP2 (FIG. 15).

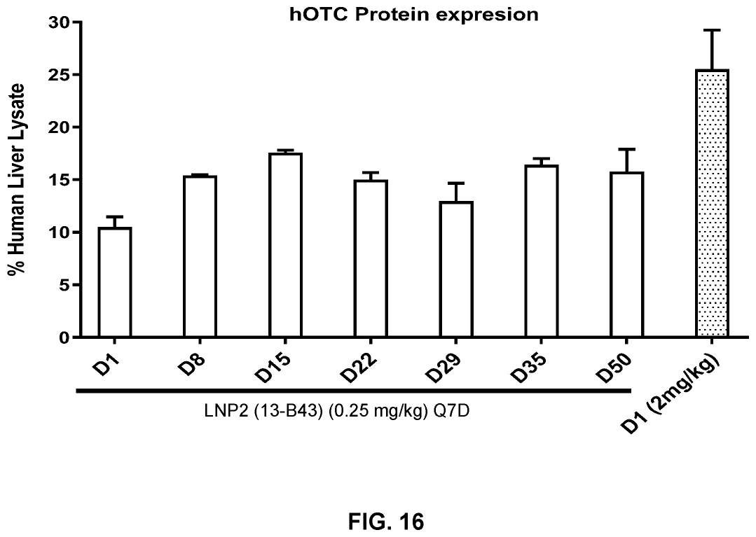

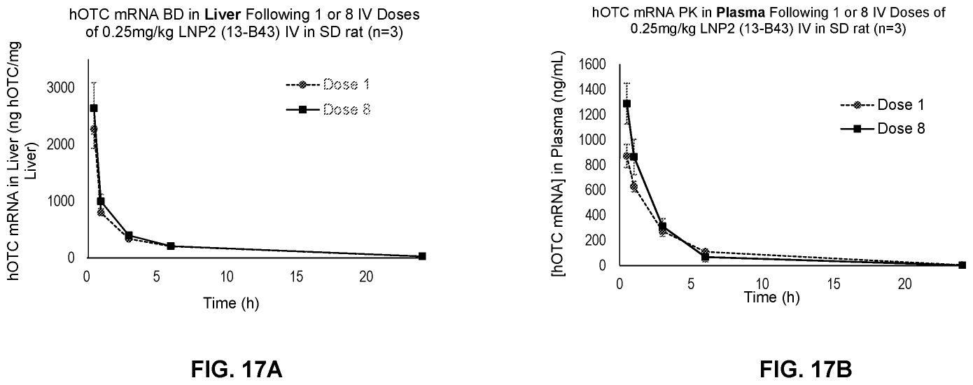

hOTC was also detected in the liver at 24 hours post every dose (FIG. 16). Levels of OTC mRNA in the liver and plasma were quantified over time (30 min, 1 h, 3 h, 6 h, and 24 h) following treatment 1 or 8 (Day 49) (FIGS. 17A and 17B).

### Example 6. Single Dose Range Finding Study in SD Rats

Next the potency and tolerability of LNP1 (PEG2000-C-DMA:13-B43:Cholesterol:DSPC), LNP2 (PEG2000-S:13-B43:Cholesterol:DSPC), and LNP2 (PEG2000-S:18-B6:Cholesterol:DSPC) formulated with OTC mRNA construct were evaluated in a dose response study with SD Rats. Rats were administered OTC mRNA construct-LNP2 at varying concentrations (0.5 mg/kg, 1 mg/kg, or 1.5 mg/kg) and analyzed at for 6 h or 24 h (Table 6A). As a control, some rats were administered 5 mL/kg PBS, 1.5 mg/kg LNP1, or 1.5 mg/kg LNP2. The Z-Avg, PDI, and % Encaps of each formulation administered is provided in Table 6B.

TABLE 6A
Dose Response Study of LNP1 and LNP2

| Group | N | Composition | Dose | |
|---|---|---|---|---|
| 1 | 3 | PBS | 5 | mL/kg |
| 2 | | LNP2 (13-B43) | 1.5 mg/kg mRNA equivalence | |
| | | | (i.e. ~30 mg/kg lipid) | |
| 3 | | LNP2 (18-B6) | 3.0 mg/kg mRNA equivalence | |
| | | | (i.e. ~60 mg/kg lipid) | |
| 4 | | LNP1 | 1.5 | mg/kg |
| 5 | | LNP2 (13-B43) | 0.5 | mg/kg |
| 6 | | | 1.0 | mg/kg |
| 7 | | | 1.5 | mg/kg |
| 8 | | LNP2 (18-B6) | 1.5 | mg/kg |
| 9 | | | 3.0 | mg/kg |

TABLE 6B
Formulation of LNPs for Dose Range Study

| | | | | | | Endotoxin |
|---|---|---|---|---|---|---|
| | | | | | | (performed |
| | | | | | | at ~1 mg/mL |
| | | | | | | mRNA; ~20 |
| LNP | | | | | | mg/mL |
| Composition | Payload | | Z-avg (nm) | PDI | Encaps (%) | lipid) |
| LNP2 (13- | Empty | PF | 62 | 0.13 | N/A | <5 EU/mL |
| B43) | | SF | 79 | 0.25 | | |
| | | F/T | 86 | 0.26 | | |
| LNP2 (18- | Empty | PF | 62 | 013 | N/A | <5 EU/mL |
| B6) | | SF | 79 | 020 | | |
| | | F/T | 83 | 0.18 | | |
| LNP1 | OTC | PF | 79 | 0.27 | 95 | <5 EU/mL |
| | mRNA | SF | 76 | 0.07 | 95 | |
| | construct | F/T | 80 | 0.08 | 98 | |
| LNP2 (13- | OTC | PF | 81 | 0.18 | 95 | <5 EU/mL |
| B43) | mRNA | SF | 83 | 0.07 | 96 | |
| | construct | F/T | 83 | 0.10 | 96 | |
| LNP2 (18- | OTC | PF | 72 | 0.10 | 94 | <5 EU/mL |
| B6) | mRNA | SF | 87 | 0.07 | 93 | |
| | construct | F/T | 86 | 0.09 | 94 | |

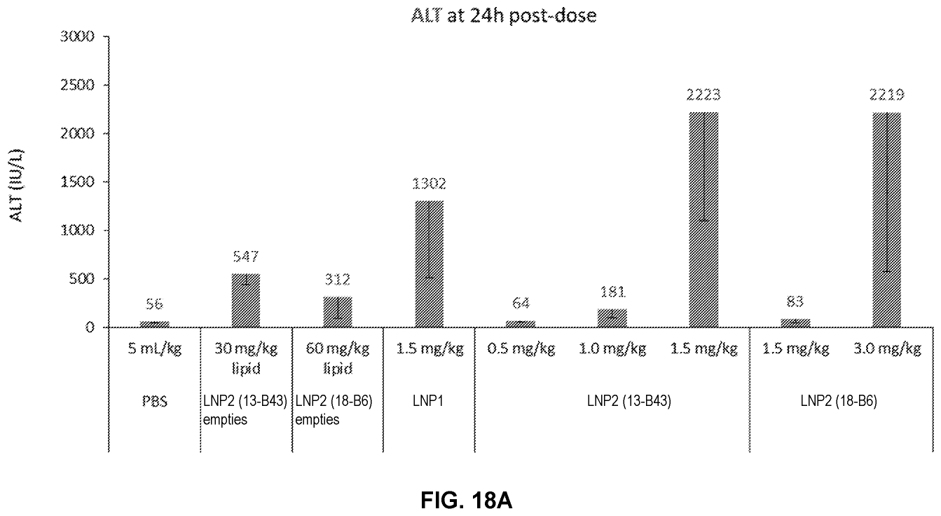

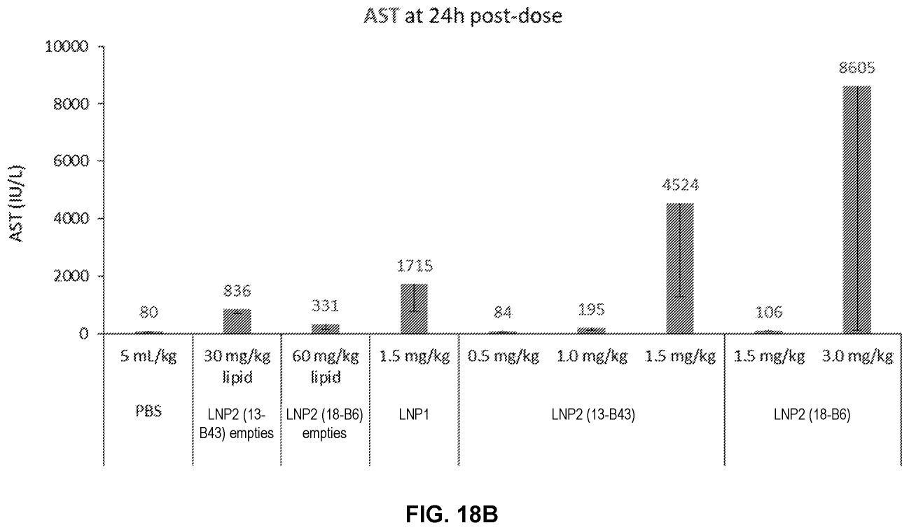

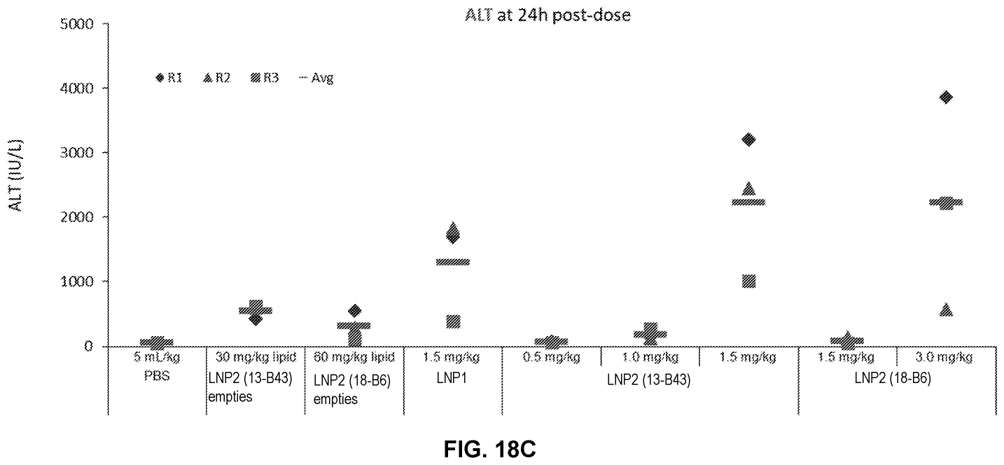

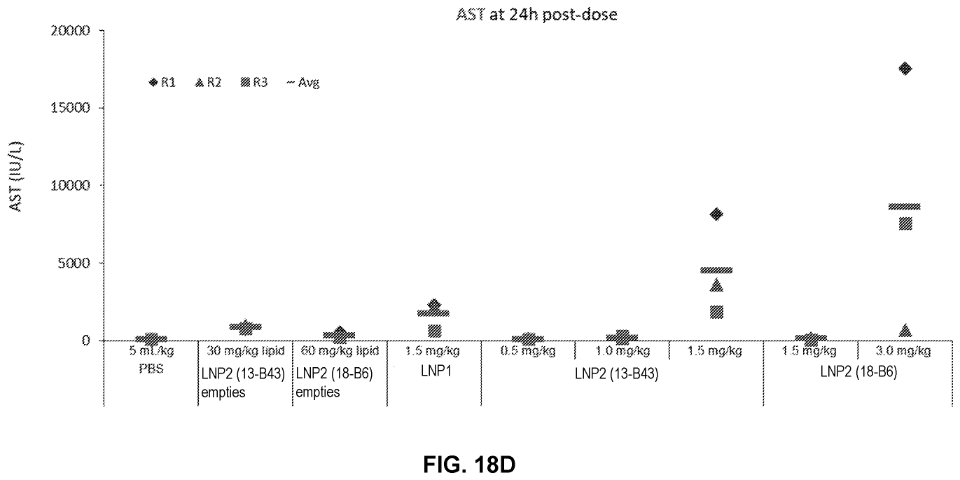

To analyze liver damage, liver samples were collected 24 h post-last dose and ALT, AST, GGT, and total bilirubin levels were analyzed. ALT/AST levels are more elevated compared with mRNA LNPs compared to empties (FIGS. 18A-18D and Table 7). There was a trend of increased ALT/AST levels with increasing dosage of LNP1, LNP2 (13-B43), or LNP2 (18-B6). Administration of 1.5 mg/kg LNP2 (13-B43) induced higher levels of ALT/AST than the same amount of LNP1.

TABLE 7
ALT and AST Levels in Rats Administered
with Different Amounts of LNP2

| Animal | Treatment | Dose | | ALT (IU/L) | AST (IU/L) |
|---|---|---|---|---|---|
| 1 | PBS | 5 | mL/kg | 52 | 71 |
| 13 | LNP2 (13- | 0.5 | mg/kg | 72 | 80 |
| 14 | B43) | | | 61 | 95 |
| 15 | | | | 59 | 76 |
| 19 | | 1.5 | mg/kg | 3211 | 8126 |
| 20 | | | | 2450 | 3597 |
| 21 | | | | 1008 | 1848 |
| 22 | LNP2 (18- | 1.5 | mg/kg | 73 | 122 |

| | | | |
|---|---|---|---|
| 23 | B6) | 129 | 126 |
| 24 | | 46 | 70 |

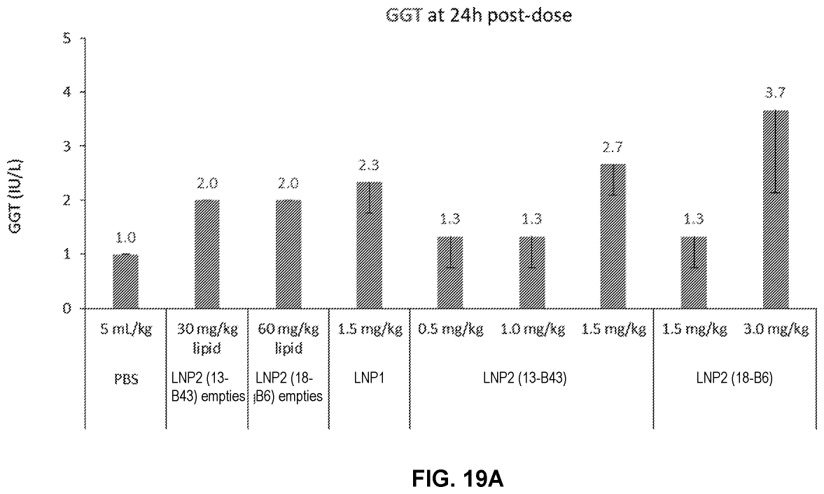

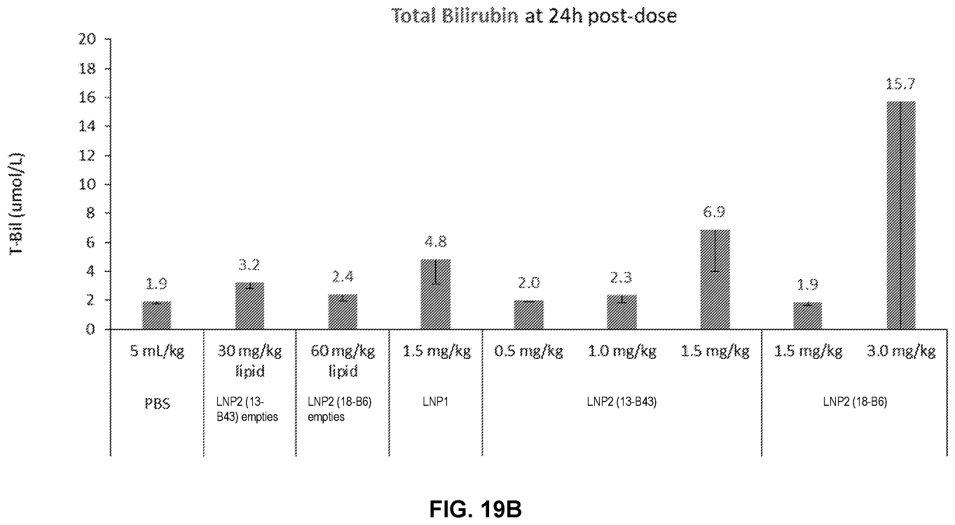

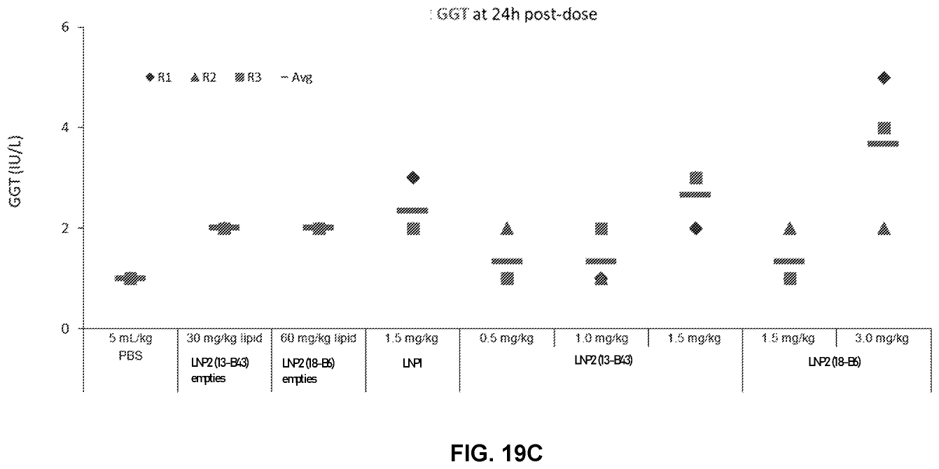

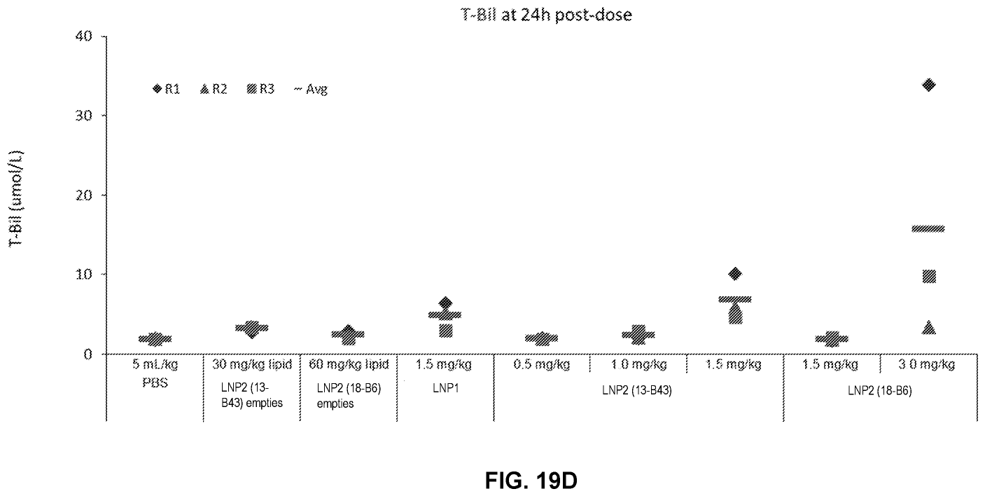

GGT and total bilirubin levels were analyzed in samples taken 24 h post last-dose. There was a trend of increased GGT and total bilirubin levels with increasing dosage of LNP1 or LNP2 OTC mRNA formulations (FIGS. 19A-19D). Administration of 1.5 mg/kg OTC mRNA construct-LNP2 (13-B43) induced similar levels of GGT compared to the same amount of OTC mRNA construct-LNP1. Administration of 1.5 mg/kg OTC mRNA construct-LNP2 induced higher levels of total bilirubin compared to the same amount of OTC mRNA construct-LNP1.

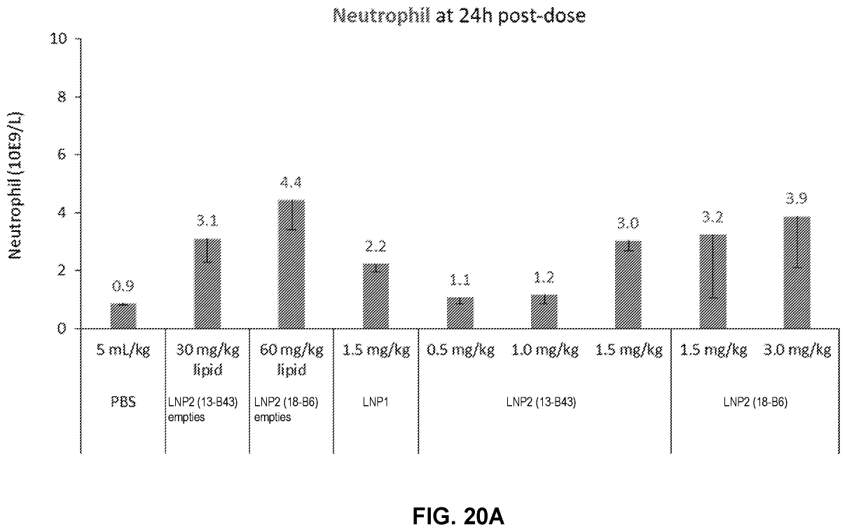

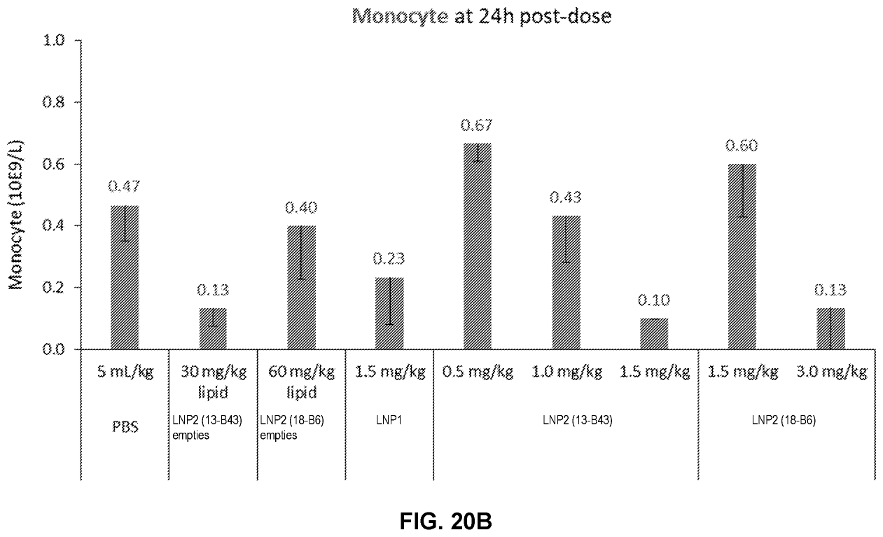

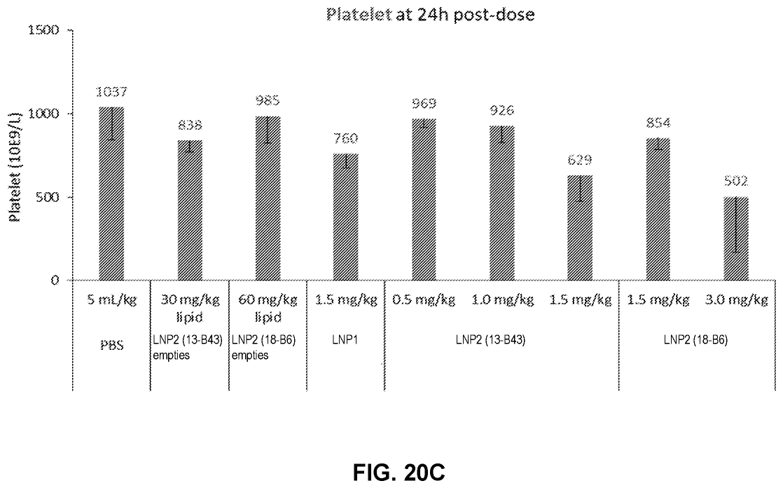

A complete blood count was obtained from blood collect 24 h post last-dose. Rats administered 1.5 mg/kg OTC mRNA construct-LNP1 had similar numbers of neutrophils, monocytes, and platelets compared with rats administered 1.5 mg/kg OTC mRNA construct-LNP2 (13-B43) (FIGS. 20A-20C). Increased dosage of OTC mRNA construct-LNP2 increased the amount of neutrophils but decreased the amount of monocytes and platelets.

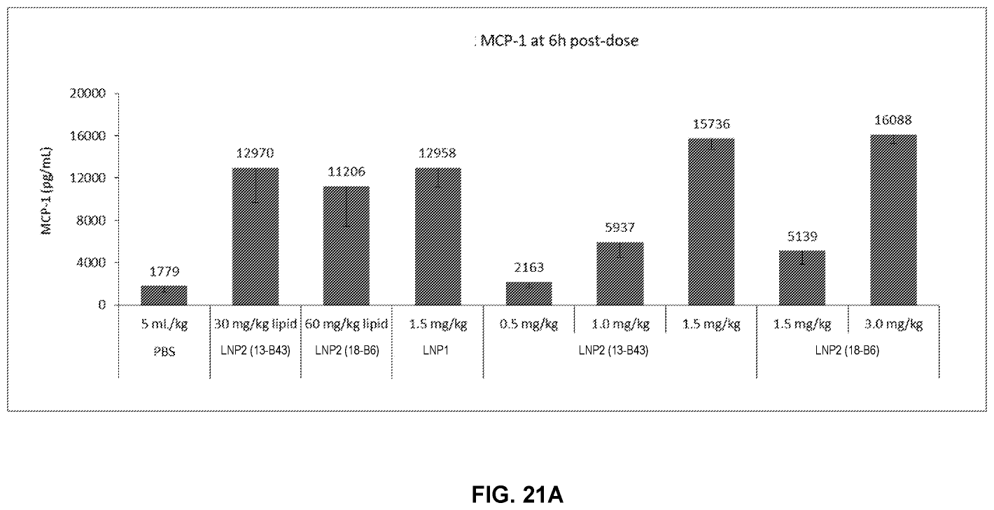

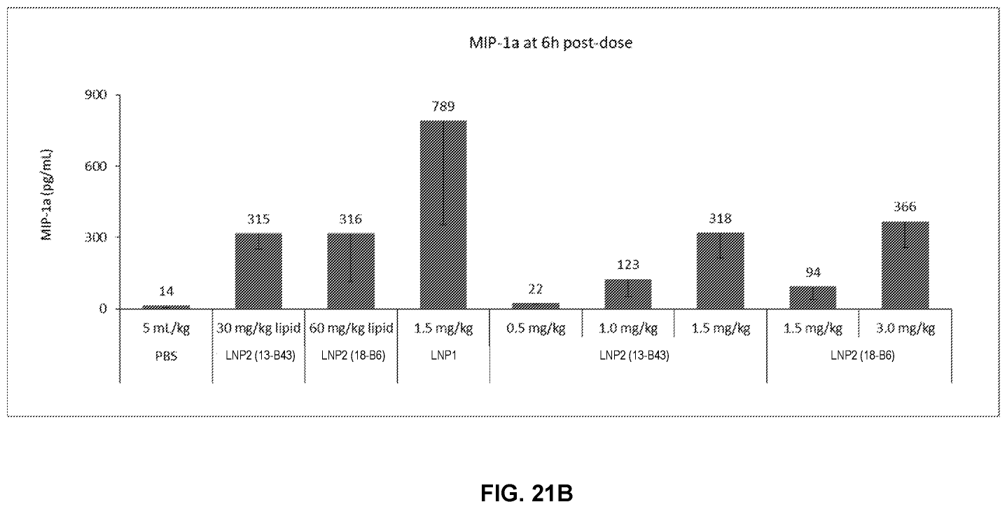

To examine cytokine levels, blood was collected 6 h post-dose and the levels of MCP-1, MIP-1α, and IP-10 were quantified. There was no significant difference in MCP-1 and MIP-la levels between empties and OTC mRNA construct-LNP compositions (FIGS. 21A-21C). The LNP1 and LNP2 OTC mRNA formulations induced higher levels of IP-10 compared to the empties. There was also a dose-dependent increase in cytokine levels with administration of OTC mRNA construct-LNP2 (13-B43).

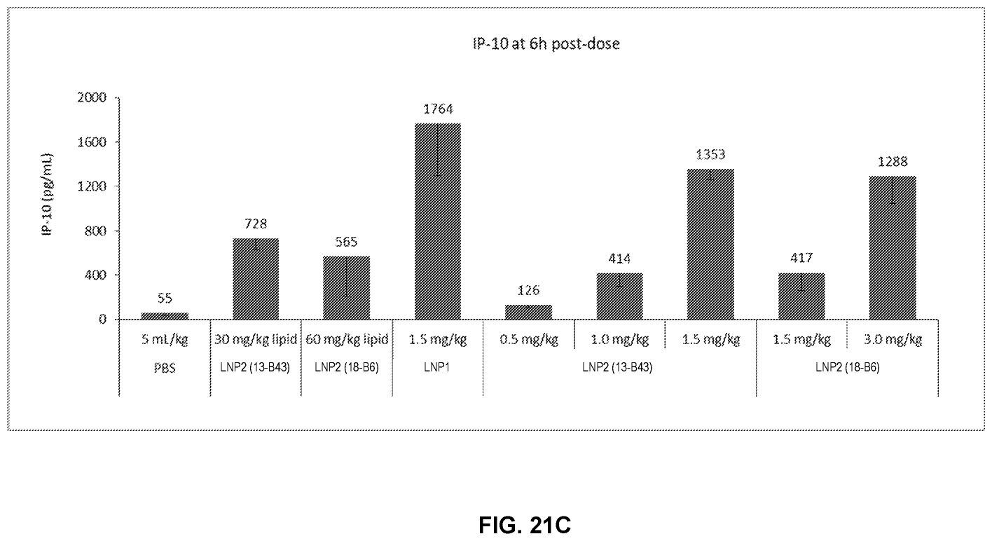

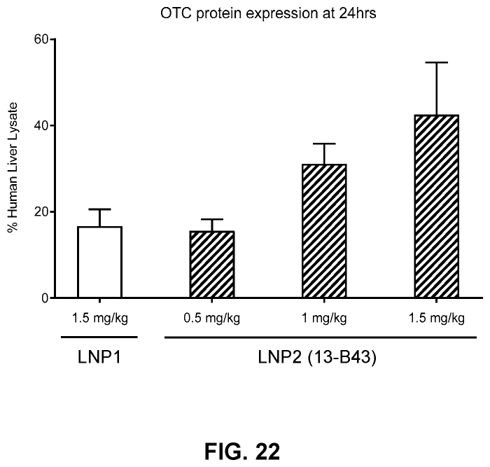

hOTC expression was examined 24 h post last-dose by western blotting. There was an dose-dependent increase in OTC expression with increasing dosage of OTC mRNA construct-LNP2 (13-B43) (FIG. 22). 1.5 mg/kg of OTC mRNA construct-LNP2 (13-B43) provided higher expression of OTC compared to 1.5 mg/kg of OTC mRNA construct-LNP1.

### Example 7. Non-Human Primate Dose Range Study

The potency of LNP1 (PEG2000-C-DMA:13-B43:Cholesterol:DSPC) formulated with OTC mRNA construct was evaluated in a dose response study in non-human primates (NHPs). The OTC mRNA construct included a nucleotide sequence having the 5′, the open reading frame, and the 3′ sequence, a polyA tail length of between 80 nucleotides to 440 nucleotides (i.e., 284 nucleotides), and was pseudouridine (v) modified. Non-human primates were administered one dose of OTC mRNA construct-LNP1 at varying concentrations (0.25 mg/kg, 1 mg/kg, 3 mg/kg, or 5 mg/kg) on three different days (day 1, 8, and 15) (Table 8). The results were analyzed at day 16. As a control, the non-human primates were administered 5 mg/kg empty LNP1.

TABLE 8
Formulation of LNPs for Non-Human Primate Dose Range Study

| Group | n | Treatment | Payload | Dose mg/kg |
|---|---|---|---|---|
| 1 | 4 | Control | — | — |
| 2 | | Empty-LNP1 | Empty | 5 (Equivalent to |
| | | | | Group 6 lipid) |
| 3 | | LNP1 | OTC mRNA | 0.25 |
| 4 | | | construct | 1 |
| 5 | | | | 3 |
| 6 | | | | 5 |

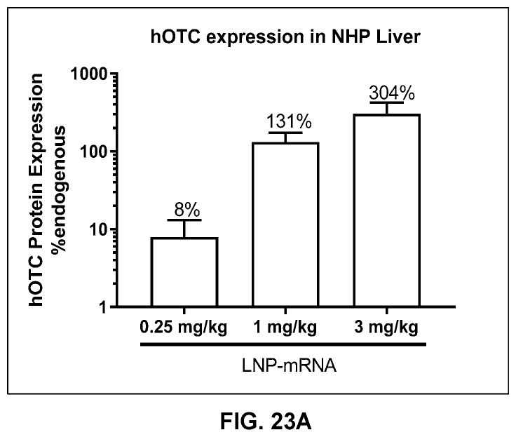

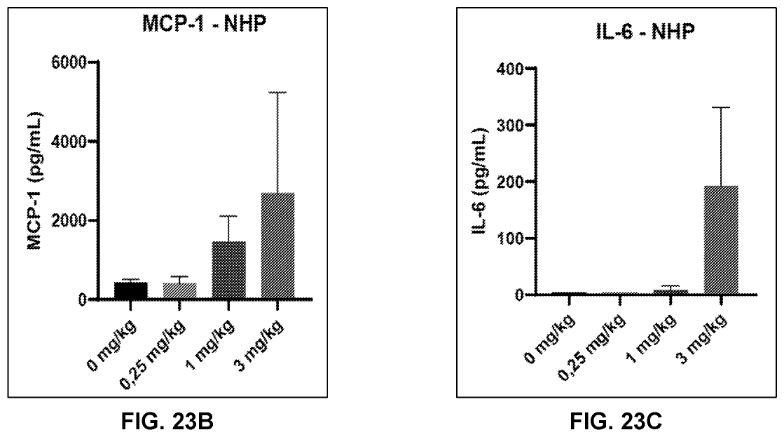

Human OTC expression was analyzed in non-human primate liver samples on day 16. The lowest OTC expression was detected with the 0.25 mg/kg dose and the highest expression was detected with the 3 mg/kg dose, relative to endogenous expression (FIG. 23A). Initial target hOTC expression (8%) was achieved at the lowest dose (0.25 mg/kg).

To examine cytokine levels, samples were collected 6 hrs after the first dose on day 1 and the level of MCP-1 and IL-6 were analyzed (FIGS. 23B & 23C). MCP-1 and IL-6 were not detected in the 0.25 mg/kg dose. Transient elevation of MCP-1 and IL-6 were observed in the 3 mg/kg dose.

These results showed strong hOTC expression with low immune stimulation.

### Example 8: In Vivo Expression of hEPO mRNA-LNP in Mice

A human erythropoietin (hEPO) polynucleotide construct was prepared by In Vitro Transcription (IVT) using a plasmid DNA construct. The plasmid DNA construct contained the instructions for the 5′UTR, ORF and 3′UTR while the chemical modification (e.g. Pseudouridine) was determined by the addition of the desired nucleotide to the IVT reaction. The mRNA was capped at the 5′ end during the IVT reaction. After the IVT reaction the resultant mRNA product was purified via LiCl precipitation and/or enzymatically polyadenylation prior to another round of purification via cellulose-based chromatography. The final mRNA product was adjusted to a desired concentration before being sterile filtered and aliquoted.

The plasmid DNA construct contained the instructions for the 5′UTR, ORF and 3′UTR. The EPO polynucleotide construct comprises a 5′UTR sequence of SEQ ID NO:1 and the 3′ UTR comprises the sequence of SEQ ID NO: 2. The sequence of the human EPO ORF is provided below:

hEPO ORF
(SEQ ID NO: 4)
AUGGGCGUGCACGAGUGCCCCGCCUGGCUGUGGCUGCUGCUGAGCCUG
CUGAGCCUGCCCCUGGGCCUGCCCGUGCUGGGCGCCCCCCCCCGGCUG
AUCUGCGACAGCCGGGUGCUGGAGCGGUACCUGCUGGAGGCCAAGGAG
GCCGAGAACAUCACCACCGGCUGCGCCGAGCACUGCAGCCUGAACGAG
AACAUCACCGUGCCCGACACCAAGGUGAACUUCUACGCCUGGAAGCGG
AUGGAGGUGGGCCAGCAGGCCGUGGAGGUGUGGCAGGGCCUGGCCCUG
CUGAGCGAGGCCGUGCUGCGGGGCCAGGCCCUGCUGGUGAACAGCAGC
CAGCCCUGGGAGCCCCUGCAGCUGCACGUGGACAAGGCCGUGAGCGGC
CUGCGGAGCCUGACCACCCUGCUGCGGGCCCUGGGCGCCCAGAAGGAG
GCCAUCAGCCCCCCCGACGCCGCCAGCGCCGCCCCCCUGCGGACCAUC
ACCGCCGACACCUUCCGGAAGCUGUUCCGGGUGUACAGCAACUUCCUG
CGGGGCAAGCUGAAGCUGUACACCGGCGAGGCCUGCCGGACCGGCGAC
CGGUGA

The human EPO (hEPO) mRNA construct was formulated into an LNP using a “T”-connector. The LNP contained 4 lipid components: PEG2000-C-DMA, 13-B43, cholesterol, and DSPC at molar ratios of 1.6:54.6:32.8:10, respectively. Lipid stocks were prepared using these lipids and molar ratios, to achieve a total concentration of ˜7 mg/mL in 100% ethanol. mRNA was diluted in acetate, pH 5 buffer and nuclease free water to achieve a target concentration of 0.366 mg/mL mRNA in 100 mM acetate, pH 5. Equal volumes of the lipid and nucleic acid solutions were blended at a flow rate of 400 mL/min through a T-connector, and diluted with ˜4 volumes of PBS, pH 7.4. Formulations were placed in Slide-A-Lyzer dialysis units (MWCO 10,000) and dialyzed overnight against 10 mM Tris, 500 mM NaCl, pH 8 buffer. Following dialysis, the formulations were concentrated to ˜ 0.6 mg/mL using VivaSpin concentrator units (MWCO 100,000) and dialyzed overnight against 5 mM Tris, 10% sucrose, pH 8 buffer. Formulations were filtered through a 0.2 μm syringe filter (PES membrane). Nucleic acid concentration was determined by the RiboGreen assay. Particle size and polydispersity were determined using a Malvern Nano Series Zetasizer.

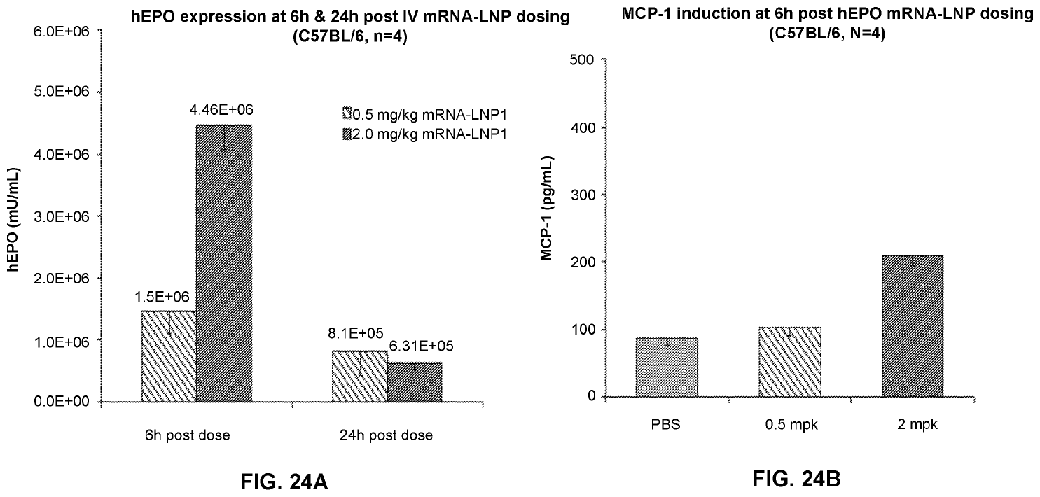

The mRNA-LNP was administered to mice intravenously. hEPO protein levels were measured in mouse plasma at 6 h and 24 h post dose. Robust expression was achieved at both the 6 h and 24 h timepoints with dose-dependent expression evident at 6 h post-dose (FIG. 24A). The tolerability of the mRNA-LNP was assessed through measurement of MCP-1 at 6 h post dose. All dose levels tested showed minimal differences compared to PBS control (FIG. 24B).

### Example 9: In Vivo Expression of hMMP-8 mRNA-LNP in Mice

A human matrix metalloproteinase 8 (hMMP-8) polynucleotide construct was prepared using the method as described in Example 8. The plasmid DNA construct contained the instructions for the 5′UTR, ORF and 3′UTR. The MMP-8 polynucleotide construct comprises a 5′UTR sequence of SEQ ID NO:1 and the 3′ UTR comprises the sequence of SEQ ID NO: 2. The sequence of the human MMP-8 ORF is provided below:

hMMP-8 ORF
(SEQ ID NO: 5)
AUGUUCAGCCUUAAAACCCUGCCUUUCCUGCUGCUGCUGCACGUGCAG
AUCUCCAAGGCCUUUCCCGUGUCCAGCAAGGAGAAGAACACCAAGACC
GUGCAGGACUACCUGGAAAAGUUCUACCAGCUGCCUAGCAAUCAAUAC
CAGAGCACCCGCAAGAACGGUACAAACGUGAUCGUGGAAAAGCUGAAG
GAAAUGCAGAGAUUCUUCGGCCUGAACGUGACAGGCAAGCCUAACGAG
GAAACACUGGACAUGAUGAAAAAGCCAAGAUGUGGCGUGCCCGACAGC
GGCGGCUUUAUGCUGACACCUGGAAAUCCUAAGUGGGAGCGGACCAAU
CUGACCUACAGAAUCCGGAACUACACCCCUCAGCUCUCCGAGGCCGAG
GUGGAAAGAGCCAUUAAGGACGCCUUCGAGCUGUGGUCCGUGGCAUCU
CCUCUGAUUUUCACCAGAAUCAGCCAGGGCGAGGCCGACAUCAACAUC
GCCUUCUACCAGCGGGACCACGGCGAUAACAGCCCCUUCGACGGCCCC
AAUGGCAUCCUCGCCCACGCCUUUCAGCCUGGACAGGGCAUAGGAGGA
GAUGCUCAUUUCGACGCUGAGGAAACCUGGACAAAUACCAGCGCCAAC
UACAACCUGUUCCUGGUGGCCGCCCACGAGUUCGGCCACAGCCUGGGC
CUGGCCCACAGCAGCGACCCCGGAGCCCUGAUGUACCCCAACUACGCC
UUCCGGGAAACAAGCAACUACAGCCUGCCUCAAGACGACAUCGACGGC
AUCCAGGCCAUCUACGGCCUGAGCUCUAACCCCAUCCAGCCUACAGGC
CCAUCUACCCCUAAGCCAUGUGAUCCUAGCCUGACCUUCGAUGCCAUC
ACGACCCUGAGAGGAGAGAUCCUGUUUUUUAAGGACAGAUACUUCUGG
CGGAGACACCCUCAGCUGCAGCGGGUUGAAAUGAACUUCAUCAGCCUG
UUCUGGCCUUCUCUGCCCACCGGCAUCCAGGCUGCCUACGAGGACUUC
GACCGGGACCUGAUCUUCCUGUUCAAGGGCAACCAGUACUGGGCCCUG
AGCGGAUAUGACAUCCUGCAGGGCUACCCUAAAGAUAUCAGCAACUAC
GGCUUCCCUAGCAGCGUGCAAGCCAUCGACGCCGCUGUGUUCUACCGG
AGCAAAACCUACUUCUUCGUGAACGACCAGUUCUGGAGAUACGACAAC
CAGAGACAGUUUAUGGAACCCGGCUAUCCUAAGAGCAUCUCUGGCGCC
UUCCCCGGCAUCGAGAGCAAGGUGGACGCCGUGUUCCAGCAGGAGCAC
UUCUUCCACGUGUUCUCUGGCCCUAGAUACUACGCCUUUGAUCUGAUC
GCCCAGAGGGUGACCAGAGUGGCCAGAGGCAACAAGUGGCUGAACUGC
AGAUAUGGCUGA

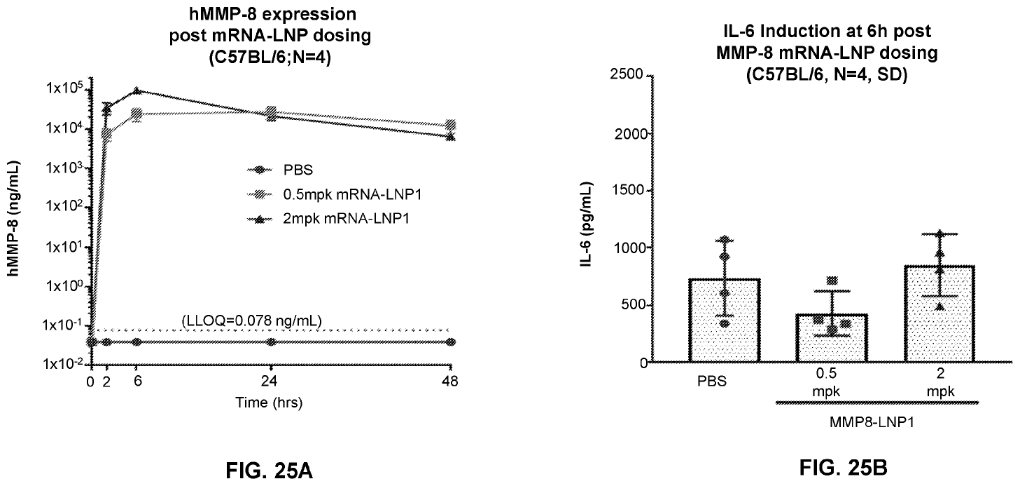

The human MMP-8 (hMMP-8) mRNA construct was formulated in the same LNP composition described in Example 8 and administered intravenously to mice. hMMP-8 protein levels were measured in mouse plasma at 0, 2-, 6-, 24- and 48-h post dose. Robust expression was achieved at all timepoints with a dose-dependent increase in expression evident at 6 h post-dose (FIG. 25A). The tolerability of the mRNA-LNP was assessed through measurement of IL-6 at 6 h post dose. All dose levels tested showed minimal differences compared to PBS control (FIG. 25B).

### Example 10: In Vivo Expression of OVA mRNA-LNP in Mice

Chicken Ovalbumin (OVA) polynucleotide constructs were synthesized using the method as described in Example 8. The plasmid DNA constructs contained the instructions for the 5′UTR, ORF and 3′UTR. The OVA polynucleotide constructs comprise a 5′UTR sequence of SEQ ID NO:1 and the 3′ UTR comprises the sequence of SEQ ID NO: 2. The sequences of the Ovalbumin ORFs (wildtype and codon modified) are provided below:

2-M9 Wildtype ORF
(SEQ ID NO: 6)
AUGGGCUCCAUCGGCGCAGCAAGCAUGGAAUUUUGUUUUGAUGUAUUCA
AGGAGCUCAAAGUCCACCAUGCCAAUGAGAACAUCUUCUACUGCCCCAU
UGCCAUCAUGUCAGCUCUAGCCAUGGUAUACCUGGGUGCAAAAGACAGC
ACCAGGACACAGAUAAAUAAGGUUGUUCGCUUUGAUAAACUUCCAGGAU
UCGGAGACAGUAUUGAAGCUCAGUGUGGCACAUCUGUAAACGUUCACUC
UUCACUUAGAGACAUCCUCAACCAAAUCACCAAACCAAAUGAUGUUUAU
UCGUUCAGCCUUGCCAGUAGACUUUAUGCUGAAGAGAGAUACCCAAUCC
UGCCAGAAUACUUGCAGUGUGUGAAGGAACUGUAUAGAGGAGGCUUGGA
ACCUAUCAACUUUCAAACAGCUGCAGAUCAAGCCAGAGAGCUCAUCAAU
UCCUGGGUAGAAAGUCAGACAAAUGGAAUUAUCAGAAAUGUCCUUCAGC
CAAGCUCCGUGGAUUCUCAAACUGCAAUGGUUCUGGUUAAUGCCAUUGU
CUUCAAAGGACUGUGGGAGAAAACAUUUAAGGAUGAAGACACACAAGCA
AUGCCUUUCAGAGUGACUGAGCAAGAAAGCAAACCUGUGCAGAUGAUGU
ACCAGAUUGGUUUAUUUAGAGUGGCAUCAAUGGCUUCUGAGAAAAUGAA
GAUCCUGGAGCUUCCAUUUGCCAGUGGGACAAUGAGCAUGUUGGUGCUG
UUGCCUGAUGAAGUCUCAGGCCUUGAGCAGCUUGAGAGUAUAAUCAACU
UUGAAAAACUGACUGAAUGGACCAGUUCUAAUGUUAUGGAAGAGAGGAA
GAUCAAAGUGUACUUACCUCGCAUGAAGAUGGAGGAAAAAUACAACCUC
ACAUCUGUCUUAAUGGCUAUGGGCAUUACUGACGUGUUUAGCUCUUCAG
CCAAUCUGUCUGGCAUCUCCUCAGCAGAGAGCCUGAAGAUAUCUCAAGC
UGUCCAUGCAGCACAUGCAGAAAUCAAUGAAGCAGGCAGAGAGGUGGUA
GGGUCAGCAGAGGCUGGAGUGGAUGCUGCAAGCGUCUCUGAAGAAUUUA
GGGCUGACCAUCCAUUCCUCUUCUGUAUCAAGCACAUCGCAACCAACGC
CGUUCUCUUCUUUGGCAGAUGUGUUUCCCCUUAA
2-M10 Codon modified ORF
(SEQ ID NO: 7)
ATGGGCAGCATTGGAGCCGCTAGCATGGAATTTTGTTTTGACGTGTTCA
AGGAACTGAAGGTCCACCACGCCAACGAGAACATCTTCTACTGCCCCAT
TGCCATTATGAGCGCCCTGGCCATGGTGTACCTGGGCGCCAAGGATTCT
ACAAGAACACAGATCAACAAGGTGGTGCGGTTCGACAAGCTGCCTGGCT
TCGGCGACAGCATCGAGGCCCAGTGCGGCACATCTGTCAACGTGCACAG
CAGCCTGCGGGACATCCTGAACCAGATCACCAAGCCCAACGACGTGTAT
AGCTTCAGCCTGGCTAGCAGACTGTACGCCGAGGAGAGATACCCTATCC
TGCCTGAGTACCTGCAATGTGTGAAGGAACTGTACAGAGGCGGACTCGA
GCCTATCAATTTCCAGACAGCCGCTGATCAGGCCAGAGAACTGATCAAC
AGCTGGGTGGAATCTCAAACCAACGGCATCATCAGAAACGTGCTGCAGC
CTAGCTCTGTGGACAGCCAGACCGCCATGGTCCTGGTGAACGCCATCGT
GTTCAAAGGACTGTGGGAGAAGACCTTCAAGGACGAAGATACCCAGGCC
ATGCCTTTCAGGGTGACCGAGCAGGAGAGCAAGCCTGTGCAGATGATGT
ACCAGATCGGCCTGTTCCGGGTGGCCAGCATGGCTTCCGAGAAGATGAA
GATCCTGGAACTGCCATTCGCCTCCGGCACCATGTCTATGCTGGTTCTG
CTGCCCGACGAGGTGTCTGGCCTTGAGCAGCTGGAAAGCATCATCAACT
TCGAGAAACTGACCGAGTGGACCAGCAGCAACGTGATGGAAGAGAGAAA
GATCAAGGTGTACCTGCCAAGAATGAAAATGGAAGAGAAGTACAACCTC
ACAAGCGTGCTGATGGCCATGGGAATCACTGATGTGTTTAGCAGCAGCG
CAAATCTGAGCGGCATCTCCTCCGCCGAGTCTCTGAAAATCAGCCAAGC
TGTGCATGCTGCCCACGCCGAGATCAATGAGGCTGGCAGAGAGGTGGTG
GGCAGCGCCGAAGCCGGCGTGGACGCCGCCTCCGTCAGCGAGGAATTCC
GGGCCGATCACCCCTTTCTGTTCTGCATCAAGCACATCGCCACCAATGC
CGTGCTGTTCTTCGGCCGGTGCGTGTCCCCTTGA

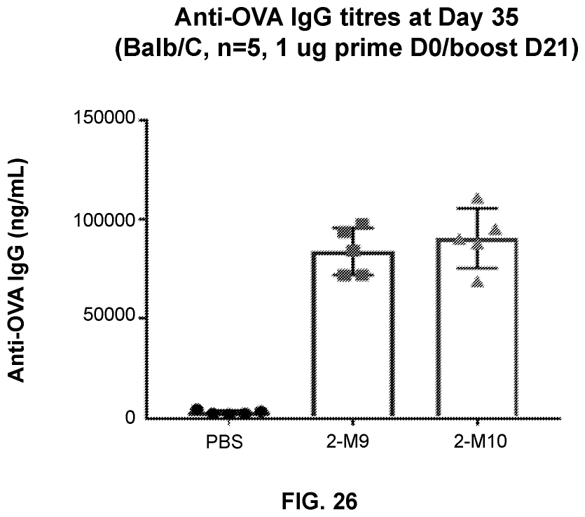

The OVA mRNA (2-M9 & 2-M10) were separately formulated in an LNP using a “T”-connector process using the method as described in Example 8. The LNP contained 4 lipid components: PEG2000-C-DMA, 13-B43, cholesterol, and DSPC at molar ratios of 1.5:50.0:38.5:10.0, respectively. A 1 μg dose of each LNP was administered intramuscularly to mice at Day 0 (DO) and Day 21 (D21). Anti-OVA IgG antibodies present in mouse plasma were quantified at Day 35 using an ELISA. Robust antibodies titers were induced by both 2-M9 and 2-M10 mRNA compared to PBS control group (FIG. 26).

### Example 11: In Vivo Expression of HA mRNA-LNP in Mice

A Hemagglutinin (HA) polynucleotide construct was synthesized using the methods as described in Example 10 and delivered intramuscularly to mice. The plasmid DNA construct contained the instructions for the 5′UTR, ORF and 3′UTR. The HA polynucleotide construct comprises a 5′UTR sequence of SEQ ID NO:1 and the 3′ UTR comprises the sequence of SEQ ID NO: 2. The HA ORF is provided below:

2-M6-HA
(SEQ ID NO: 8)
ATGAAGGCGAACCTGCTGGTCCTGCTGAGCGCGCTGGCGGCGGCGGACG
CGGACACGATCTGCATCGGCTACCACGCGAACAACAGCACCGACACGGT
CGACACGGTCCTCGAGAAGAACGTGACCGTGACCCACAGCGTCAACCTG
CTCGAGGACAGCCACAACGGGAAGCTGTGCAGGCTCAAGGGCATCGCCC
CGCTGCAGCTGGGGAAGTGCAACATCGCCGGCTGGCTCTTGGGGAACCC
CGAGTGCGACCCGCTGCTCCCGGTGAGGAGCTGGTCCTACATCGTGGAG
ACCCCGAACTCGGAGAACGGGATCTGCTACCCGGGGGACTTCATCGACT
ACGAGGAGCTGAGGGAGCAGTTGAGCTCGGTGTCGTCCTTCGAGAGGTT
CGAGATCTTCCCCAAGGAGAGCTCGTGGCCCAACCACAACACCAACGGG
GTCACGGCCGCGTGCTCCCACGAGGGGAAGAGCAGCTTCTACAGGAACT
TGCTGTGGCTGACGGAGAAGGAGGGCTCGTACCCGAAGCTGAAGAACTC
GTACGTGAACAAGAAGGGGAAGGAGGTCCTCGTACTGTGGGGCATCCAC
CACCCGCCGAACAGCAAGGAGCAGCAGAACCTCTACCAGAACGAGAATG
CGTACGTCTCCGTGGTGACCTCGAACTACAACAGGAGGTTCACCCCGGA
GATCGCGGAGAGGCCCAAGGTCAGGGACCAGGCCGGGAGGATGAACTAC
TACTGGACCTTGCTGAAGCCCGGCGACACCATCATCTTCGAGGCGAACG
GGAACCTGATCGCACCGATGTATGCGTTCGCGCTGAGCAGGGGCTTCGG
GTCCGGCATCATCACCTCGAATGCGTCCATGCACGAGTGCAACACGAAG
TGCCAGACGCCCCTGGGCGCGATCAACAGCAGCCTCCCGTACCAGAACA
TCCACCCGGTCACGATCGGGGAGTGCCCCAAGTACGTCAGGAGCGCCAA
GTTGAGGATGGTGACCGGGCTCAGGAACACGCCGTCCATCCAGTCCAGG
GGCCTGTTCGGGGCCATCGCCGGGTTCATCGAGGGGGGCTGGACCGGCA
TGATCGACGGGTGGTACGGGTACCACCACCAGAACGAGCAGGGGTCGGG
CTACGCGGCGGACCAGAAGAGCACGCAGAACGCCATCAACGGGATCACG
AACAAGGTGAACACGGTCATCGAGAAGATGAACATCCAGTTCACGGCCG
TGGGGAAGGAGTTCAACAAGTTGGAGAAGAGGATGGAGAACTTGAACAA
GAAGGTCGACGACGGGTTCCTGGACATCTGGACGTACAACGCGGAGTTG
TTGGTGCTGCTGGAGAACGAGAGGACGCTGGACTTCCACGACTCGAACG
TGAAGAACCTGTACGAGAAGGTGAAGAGCCAGTTGAAGAACAACGCCAA
GGAGATCGGCAACGGGTGCTTCGAGTTCTACCACAAGTGCGACAACGAG
TGCATGGAGAGCGTGAGGAACGGGACGTACGACTACCCCAAGTACTCCG
AAGAGTCGAAGTTGAACAGGGAGAAGGTGGACGGGGTGAAGTTGGAGTC
GATGGGGATCTACCAGATCCTGGCGATCTACTCGACGGTCGCCAGCTCC
CTGGTGCTGTTGGTCTCCCTGGGGGCGATCAGCTTCTGGATGTGCTCCA
ACGGGTCGTTGCAGTGCAGGATCTGCATCTGA

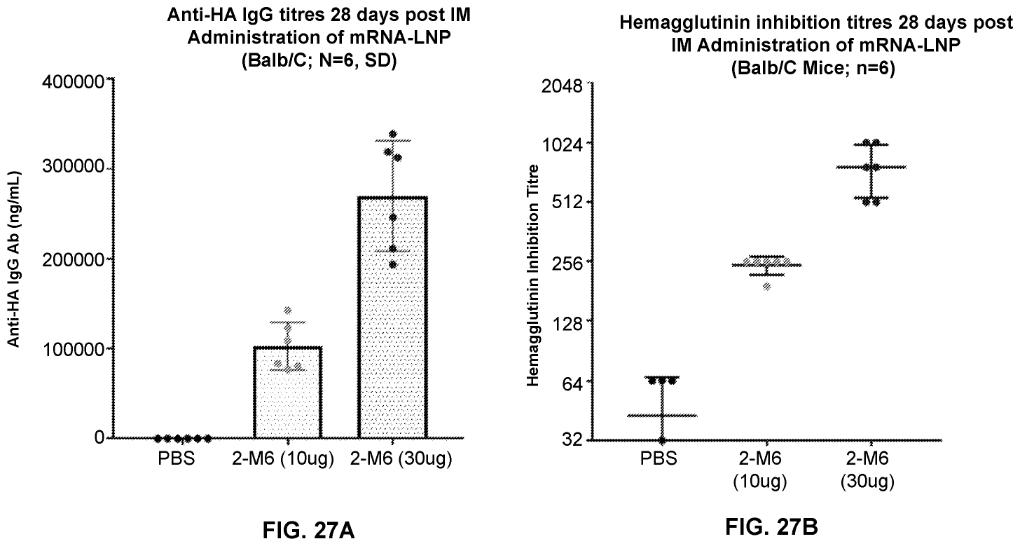

The HA mRNA (2-M6) was formulated using a T-connector process and the same LNP composition as described in Example 10, then administered to mice. A 10 μg or 30 μg dose was administered at Day 0 (DO). Anti-HA IgG antibodies present in mouse serum were quantified at Day 28. Robust antibodies titers were induced by 2-M6 in a dose dependent manner (FIG. 27A). Hemagglutinin inhibition titers were also measured using serum from the mice taken at Day 28 (FIG. 27B). It is evident that mice treated with HA mRNA-LNP displayed higher titers compared to PBS control animals.

Thus, various aspects are disclosed. The implementations described above and other implementations are within the scope of the following claims. One skilled in the art will appreciate that the present disclosure can be practiced with aspects other than those disclosed. The disclosed aspects are presented for purposes of illustration and not limitation, and the present disclosure is limited only by the claims that follow.

We claim:

1. A polynucleotide construct comprising, from 5′ to 3′:
(a) a 5′ UTR comprising a sequence at least 95% identical to the sequence of SEQ ID NO: 1;
(b) an mRNA sequence comprising an open reading frame (ORF) encoding a functional protein of interest; and
(c) a 3′ UTR comprising a sequence at least 95% identical to the sequence of SEQ ID NO: 2.

2. The polynucleotide construct of claim 1, wherein the 5′ UTR comprises the sequence of SEQ ID NO: 1.

3. The polynucleotide construct of claim 1 or 2, wherein the 3′ UTR comprises the sequence of SEQ ID NO: 2.

4. The polynucleotide construct of any one of claims 1-3 which further comprises a 5′ terminal cap.

5. The polynucleotide construct of claim 4, wherein the 5′ terminal cap is a Cap1.

6. The polynucleotide construct of any one of claims 1-5, which further comprises a polyA tail.

7. The polynucleotide construct of claim 6, wherein the polyA tail is between 80 and 1000 nucleic acids long.

8. The polynucleotide construct of claim 6, wherein polyA tail is between 100 and 500 nucleic acids long.

9. A polynucleotide construct comprising, from 5′ to 3′:
(a) a 5′ terminal cap;
(b) a 5′ UTR comprising a sequence at least 99% identical to the sequence of SEQ ID NO: 1;
(c) an mRNA sequence comprising an open reading frame (ORF) encoding a functional protein of interest;
(d) a 3′ UTR comprising a sequence at least 99% identical to the sequence of SEQ ID NO: 2; and
(e) a polyA tail that is between 100 and 500 nucleic acids long.

10. The polynucleotide construct of claim 9, wherein the 5′ UTR comprises the sequence of SEQ ID NO: 1 and the 3′ UTR comprises the sequence of SEQ ID NO: 2.

11. The polynucleotide construct of any one of claims 1-10, wherein the mRNA comprises at least one chemically modified uridine.

12. The polynucleotide construct of claim 11, wherein at least about 50%, at least about 60%, at least about 70%, at least about 80%, at least about 85%, at least about 90%, at least about 95%, or about 100% of the uridines are chemically modified.

13. The polynucleotide construct of claim 11 or 12, wherein the chemically modified uridine is selected from the group consisting of pseudouridine (w), N1-methyl pseudouridine (N1-me-ψ), and a combination thereof.

14. A composition comprising:
(a) a polynucleotide construct of any one of claims 1-13; and
(b) a delivery agent.

15. The composition of claim 14, wherein the delivery agent comprises a lipid nanoparticle (LNP), a liposome, a polymer, a micelle, a plasmid, a virus, or any combination thereof.

16. The composition of claim 15, wherein the LNP is selected from the group consisting of PEG2000-C-DMA:13-B43:Cholesterol:DSPC, PEG2000-S:13-B43:Cholesterol:DSPC, PEG2000-S:18-B6:Cholesterol:DSPC, and PEG750-C-DLA:18-B6:Cholesterol:DSPC.

17. The composition of claim 15 or 16, wherein the polynucleotide construct is encapsulated in the LNP.

18. The composition of claim 17, wherein the polynucleotide construct is fully encapsulated in the LNP.

19. The composition of claim 18, wherein at least 95% of the polynucleotide construct is encapsulated in the LNP.

20. The composition of any one of claims 14-19, which further comprises a pharmaceutically acceptable carrier.

21. A method for increasing the expression of a protein of interest in a cell comprising administering to the cell a composition comprising the polynucleotide construct of any one of claims 1-13 or the composition of any one of claims 14-20.

22. A method for treating or reducing the symptoms associated with a disease or disorder comprising administering to a subject in need thereof a therapeutically effective amount of a composition comprising the polynucleotide construct of any one of claims 1-13 or the composition of any one of claims 14-20.

23. An expression cassette comprising the polynucleotide construct of any one of claims 1-13 and 14-20.

24. The expression cassette of claim 23, which further comprises a promoter.

25. The expression cassette of claim 24, wherein the promoter is a T7 promoter.

26. A plasmid comprising the expression cassette of any one of claims 23-25.

27. A host cell comprising the expression cassette of any one of claims 23-25 or the plasmid of claim 26.

28. Use of the polynucleotide construct of any one of claims 1-13, or the composition of any one of claims 14-20, the expression cassette of claim any one of claims 23-25, the plasmid of claim 26, or the host cell of claim 27, for the manufacture of a medicament for the treatment of a disease or disorder in a subject in need thereof.

29. A method for the in vivo delivery of a nucleic acid, the method comprising:
administering to a mammalian subject the polynucleotide construct of any one of claims 1-13, the composition of any one of claims 14-20, the expression cassette of any one of claims 23-25, the plasmid of claim 26, or the host cell of claim 27.

30. A method for treating a disease or disorder in a mammalian subject in need thereof, the method comprising: administering to the mammalian subject a therapeutically effective amount of the polynucleotide construct of any one of claims 1-13, the composition of any one of claims 14-20, the expression cassette of any one of claims 23-25, the plasmid of claim 26, or the host cell of claim 27.

31. The method of claim 30, wherein the disease or disorder is a genetic disease or disorder.

32. The method of claim 30, wherein the disease or disorder is an infectious disease or a cancer.

33. The use or method of any one of claims 28-32, wherein the functional protein of interest comprises an enzyme, a growth factor, a cytokine, a receptor, a receptor ligand, a hormone, a membrane protein, a membrane-associated protein, an antigen, or an antibody.


SEQUENCE LISTING (8 sequences)

SEQ ID NO: 1 - RNA, 47 nt, Artificial Sequence
  5′ UTR
aggaaauaagagagaaaagaagaguaagaagaaauauaagagccacc

SEQ ID NO: 2 - RNA, 114 nt, Artificial Sequence
  3′ UTR
gcggccgcuuaauuaagcugccuucugcggggcuugccuucuggccaugcccuucuucucucccuugcaccuguaccucuuggucuuugaauaaagccugaguaggaagucuag

SEQ ID NO: 3 - DNA, 17 bp, Artificial Sequence
  T7 Promoter
taatacgactcactata

SEQ ID NO: 4 - RNA, 582 nt, Artificial Sequence
  hEPO ORF
gacagccgggugcuggagcgguaccugcuggaggccaaggaggccgagaacaucaccaccggcugcgccgagcacugcagccugaacgagaacaucaccgugcccgacaccaaggugaacuucuacgccuggaagcggauggaggugggccagcaggccguggagguguggcagggccuggcccugcugagcgaggccgugcugcggggccaggcccugcuggugaacagcagccagcccugggagccccugcagcugcacguggacaaggccgugagcggccugcggagccugaccacccugcugcgggcccugggcgcccagaaggaggccaucagcccccccgacgccgccagcgccgccccccugcggaccaucaccgccgacaccuuccggaagcuguuccggguguacagcaacuuccugcggggcaagcugaagcuguacaccggcgaggccugccggaccggcgaccggugaaugggcgugcacgagugccccgccuggcuguggcugcugcugagccugcugagccugccccugggccugcccgugcugggcgcccccccccggcugaucugc

SEQ ID NO: 5 - RNA, 1404 nt, Artificial Sequence
  hMMP-8 ORF
auguucagccuuaaaacccugccuuuccugcugcugcugcacgugcagaucuccaaggccuuucccguguccagcaaggagaagaacaccaagaccgugcaggacuaccuggaaaaguucuaccagcugccuagcaaucaauaccagagcacccgcaagaacgguacaaacgugaucguggaaaagcugaaggaaaugcagagauucuucggccugaacgugacaggcaagccuaacgaggaaacacuggacaugaugaaaaagccaagauguggcgugcccgacagcggcggcuuuaugcugacaccuggaaauccuaagugggagcggaccaaucugaccuacagaauccggaacuacaccccucagcucuccgaggccgagguggaaagagccauuaaggacgccuucgagcugugguccguggcaucuccucugauuuucaccagaaucagccagggcgaggccgacaucaacaucgccuucuaccagcgggaccacggcgauaacagccccuucgacggccccaauggcauccucgcccacgccuuucagccuggacagggcauaggaggagaugcucauuucgacgcugaggaaaccuggacaaauaccagcgccaacuacaaccuguuccugguggccgcccacgaguucggccacagccugggccuggcccacagcagcgaccccggagcccugauguaccccaacuacgccuuccgggaaacaagcaacuacagccugccucaagacgacaucgacggcauccaggccaucuacggccugagcucuaaccccauccagccuacaggcccaucuaccccuaagccaugugauccuagccugaccuucgaugccaucacgacccugagaggagagauccuguuuuuuaaggacagauacuucuggcggagacacccucagcugcagcggguugaaaugaacuucaucagccuguucuggccuucucugcccaccggcauccaggcugccuacgaggacuucgaccgggaccugaucuuccuguucaagggcaaccaguacugggcccugagcggauaugacauccugcagggcuacccuaaagauaucagcaacuacggcuucccuagcagcgugcaagccaucgacgccgcuguguucuaccggagcaaaaccuacuucuucgugaacgaccaguucuggagauacgacaaccagagacaguuuauggaacccggcuauccuaagagcaucucuggcgccuuccccggcaucgagagcaagguggacgccguguuccagcaggagcacuucuuccacguguucucuggcccuagauacuacgccuuugaucugaucgcccagagggugaccagaguggccagaggcaacaaguggcugaacugcagauauggcuga

SEQ ID NO: 6 - RNA, 1161 nt, Artificial Sequence
  2-M9 Wildtype ORF
augggcuccaucggcgcagcaagcauggaauuuuguuuugauguauucaaggagcucaaaguccaccaugccaaugagaacaucuucuacugccccauugccaucaugucagcucuagccaugguauaccugggugcaaaagacagcaccaggacacagauaaauaagguuguucgcuuugauaaacuuccaggauucggagacaguauugaagcucaguguggcacaucuguaaacguucacucuucacuuagagacauccucaaccaaaucaccaaaccaaaugauguuuauucguucagccuugccaguagacuuuaugcugaagagagauacccaauccugccagaauacuugcagugugugaaggaacuguauagaggaggcuuggaaccuaucaacuuucaaacagcugcagaucaagccagagagcucaucaauuccuggguagaaagucagacaaauggaauuaucagaaauguccuucagccaagcuccguggauucucaaacugcaaugguucugguuaaugccauugucuucaaaggacugugggagaaaacauuuaaggaugaagacacacaagcaaugccuuucagagugacugagcaagaaagcaaaccugugcagaugauguaccagauugguuuauuuagaguggcaucaauggcuucugagaaaaugaagauccuggagcuuccauuugccagugggacaaugagcauguuggugcuguugccugaugaagucucaggccuugagcagcuugagaguauaaucaacuuugaaaaacugacugaauggaccaguucuaauguuauggaagagaggaagaucaaaguguacuuaccucgcaugaagauggaggaaaaauacaaccucacaucugucuuaauggcuaugggcauuacugacguguuuagcucuucagccaaucugucuggcaucuccucagcagagagccugaagauaucucaagcuguccaugcagcacaugcagaaaucaaugaagcaggcagagaggugguagggucagcagaggcuggaguggaugcugcaagcgucucugaagaauuuagggcugaccauccauuccucuucuguaucaagcacaucgcaaccaacgccguucucuucuuuggcagauguguuuccccuuaa

SEQ ID NO: 7 - DNA, 1161 bp, Artificial Sequence
  2-M10 Codon optimized ORF
atgggcagcattggagccgctagcatggaattttgttttgacgtgttcaaggaactgaaggtccaccacgccaacgagaacatcttctactgccccattgccattatgagcgccctggccatggtgtacctgggcgccaaggattctacaagaacacagatcaacaaggtggtgcggttcgacaagctgcctggcttcggcgacagcatcgaggcccagtgcggcacatctgtcaacgtgcacagcagcctgcgggacatcctgaaccagatcaccaagcccaacgacgtgtatagcttcagcctggctagcagactgtacgccgaggagagataccctatcctgcctgagtacctgcaatgtgtgaaggaactgtacagaggcggactcgagcctatcaatttccagacagccgctgatcaggccagagaactgatcaacagctgggtggaatctcaaaccaacggcatcatcagaaacgtgctgcagcctagctctgtggacagccagaccgccatggtcctggtgaacgccatcgtgttcaaaggactgtgggagaagaccttcaaggacgaagatacccaggccatgcctttcagggtgaccgagcaggagagcaagcctgtgcagatgatgtaccagatcggcctgttccgggtggccagcatggcttccgagaagatgaagatcctggaactgccattcgcctccggcaccatgtctatgctggttctgctgcccgacgaggtgtctggccttgagcagctggaaagcatcatcaacttcgagaaactgaccgagtggaccagcagcaacgtgatggaagagagaaagatcaaggtgtacctgccaagaatgaaaatggaagagaagtacaacctcacaagcgtgctgatggccatgggaatcactgatgtgtttagcagcagcgcaaatctgagcggcatctcctccgccgagtctctgaaaatcagccaagctgtgcatgctgcccacgccgagatcaatgaggctggcagagaggtggtgggcagcgccgaagccggcgtggacgccgcctccgtcagcgaggaattccgggccgatcacccctttctgttctgcatcaagcacatcgccaccaatgccgtgctgttcttcggccggtgcgtgtccccttga

SEQ ID NO: 8 - DNA, 1698 bp, Artificial Sequence
  2-M6-HA
atgaaggcgaacctgctggtcctgctgagcgcgctggcggcggcggacgcggacacgatctgcatcggctaccacgcgaacaacagcaccgacacggtcgacacggtcctcgagaagaacgtgaccgtgacccacagcgtcaacctgctcgaggacagccacaacgggaagctgtgcaggctcaagggcatcgccccgctgcagctggggaagtgcaacatcgccggctggctcttggggaaccccgagtgcgacccgctgctcccggtgaggagctggtcctacatcgtggagaccccgaactcggagaacgggatctgctacccgggggacttcatcgactacgaggagctgagggagcagttgagctcggtgtcgtccttcgagaggttcgagatcttccccaaggagagctcgtggcccaaccacaacaccaacggggtcacggccgcgtgctcccacgaggggaagagcagcttctacaggaacttgctgtggctgacggagaaggagggctcgtacccgaagctgaagaactcgtacgtgaacaagaaggggaaggaggtcctcgtactgtggggcatccaccacccgccgaacagcaaggagcagcagaacctctaccagaacgagaatgcgtacgtctccgtggtgacctcgaactacaacaggaggttcaccccggagatcgcggagaggcccaaggtcagggaccaggccgggaggatgaactactactggaccttgctgaagcccggcgacaccatcatcttcgaggcgaacgggaacctgatcgcaccgatgtatgcgttcgcgctgagcaggggcttcgggtccggcatcatcacctcgaatgcgtccatgcacgagtgcaacacgaagtgccagacgcccctgggcgcgatcaacagcagcctcccgtaccagaacatccacccggtcacgatcggggagtgccccaagtacgtcaggagcgccaagttgaggatggtgaccgggctcaggaacacgccgtccatccagtccaggggcctgttcggggccatcgccgggttcatcgaggggggctggaccggcatgatcgacgggtggtacgggtaccaccaccagaacgagcaggggtcgggctacgcggcggaccagaagagcacgcagaacgccatcaacgggatcacgaacaaggtgaacacggtcatcgagaagatgaacatccagttcacggccgtggggaaggagttcaacaagttggagaagaggatggagaacttgaacaagaaggtcgacgacgggttcctggacatctggacgtacaacgcggagttgttggtgctgctggagaacgagaggacgctggacttccacgactcgaacgtgaagaacctgtacgagaaggtgaagagccagttgaagaacaacgccaaggagatcggcaacgggtgcttcgagttctaccacaagtgcgacaacgagtgcatggagagcgtgaggaacgggacgtacgactaccccaagtactccgaagagtcgaagttgaacagggagaaggtggacggggtgaagttggagtcgatggggatctaccagatcctggcgatctactcgacggtcgccagctccctggtgctgttggtctccctgggggcgatcagcttctggatgtgctccaacgggtcgttgcagtgcaggatctgcatctga